(19) United States
(12) Patent Application Publication
(10) Pub. No.: US 2009/0088482 A1
(43) Pub. Date: Apr. 2, 2009

(54) Compositions and Methods For Treating Heart Failure

(75) Inventors: Simon Maybaum, White Plains, NY (US); Jie Wang, Englewood Cliffs, NJ (US); Mehmet C. Oz, Cliffside Park, NJ (US); Steve Xydas, Ardsley, NY (US); Aftab Kherani, New York, NY (US)

Correspondence Address: WilmerHale/Columbia University, 399 PARK AVENUE, NEW YORK, NY, 10022, US

(73) Assignee: The Trustees Of Columbia University In The City New York, New York, NY (US)

(21) Appl. No.: 11/885,391

(22) PCT Filed: Oct. 13, 2004
(86) PCT No.: PCT/US04/34426

§ 371 Date: Nov. 12, 2008

Related U.S. Application Data
(60) Provisional application No. 60/511,619, filed on Oct. 14, 2003.
(60) Provisional application No. 60/549,803, filed on Mar. 2, 2004.

(51) Int. Cl. A61K 31/138

(52) U.S. Cl. 514/653

(57) ABSTRACT

The present invention provides compositions and methods for the following: preventing and treating heart failure; preventing heart failure in a patient with a pre-heart failure condition; treating and preventing heart failure with ischemic and non-ischemic causes; treating and preventing heart failure in a subject status post myocardial infarction; reversing damage to the heart following myocardial infarction; by administering to a subject an effective amount of an adrenergic beta-agonist either alone or in combination with an effective amount of an adrenergic beta-1 antagonist. FIG. 1 is a bar graph representing relative infarct sizes, as expressed as a percentage of left ventricular circumference.

This application claims the benefit of U.S. provisional applications U.S. Ser. No. 60/511,619, filed Oct. 13, 2003 and U.S. Ser. No. 60/549,803, filed Mar. 2, 2004, which are hereby incorporated by reference into the subject application in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### COPYRIGHT NOTICE

A portion of the disclosure of this patent document contains or may contain material, which is subject to copyright protection. The copyright owner has no objection to the photocopy reproduction by anyone of the patent document or the patent disclosure in exactly the form it appears in the Patent and Trademark Office patent file or records, but otherwise reserves all copyright rights whatsoever.

### BACKGROUND OF THE INVENTION

Heart failure is a leading cause of mortality and morbidity worldwide. In the United States it affects nearly 5 million people and is the only major cardiovascular disorder on the rise. It is estimated that 400,000 to 700,000 new cases of heart failure are diagnosed each year in the U.S. and the number of deaths in the U.S. attributable to this condition has more than doubled since 1979, currently averaging 250,000 annually. (Heart Failure Association of America). Less than 50 percent of patients survive five years after their initial diagnosis of heart failure, and less than 25 percent are alive 10 years after their initial diagnosis. (Heart Failure Association of America). In the more severe cases of heart failure (New York Heart Association class IV), the 2-year mortality rate is over 50% (Braunwald, E. B., Heart Disease, 4th ed. (Philadelphia: W.B. Saunders Co., 1992)). Although heart failure affects people of all ages, the risk of heart failure increases with age and is most common among older people. Accordingly, the number of people living with heart failure is expected to increase significantly as the elderly population grows over the next few decades. The causes of heart failure have been linked to various disorders including coronary artery disease, past myocardial infarction, hypertension, abnormal heart valves, cardiomyopathy or myocarditis, congenital heart disease, severe lung disease, diabetes, severe anemia, hyperthyroidism, arrhythmia or dysrhythmia.

Heart failure, also called congestive heart failure (CHF), is commonly characterized by decreased cardiac output, decreased cardiac contractility, abnormal diastolic compliance, reduced stroke volume, and pulmonary congestion. The clinical manifestations of heart failure reflect a decrease in the myocardial contractile state and a reduction in cardiac output. Apart from deficiencies in cardiac contractility, the CHF disease state may arise from left ventricular failure, right ventricular failure, biventricular failure, systolic dysfunction, diastolic dysfunction, and pulmonary effects. A progressive decrease in the contractile function of cardiac muscle, associated with heart disease, often leads to hypoperfusion of critical organs.

For example, in CHF, plasma volume may increase, causing fluid to accumulate in the lungs, abdominal organs, and peripheral tissues (Beers and Berkow, eds., The Merck Manual of Diagnosis and Therapy, 17th ed. (Whitehouse Station, N.J.: Merck Research Laboratories, 1999) 1682-88). Cardiac arrhythmia, another common feature of heart failure, results in many of the deaths associated with the disease.

Although symptoms and effects within each category may differ from individual to individual, there are two main categories of heart failure, systolic heart failure and diastolic heart failure. Systolic heart failure is characterized by a decrease in the heart's ability to contract with sufficient force, resulting in the heart's inability to push enough blood into circulation. In comparison, diastolic failure is characterized by a stiffening of the heart muscle. This decrease in the heart's ability to relax, results in the heart's failure to properly fill with blood during the resting period between each beat.

Drug treatment for heart failure primarily involves diuretics, ACE inhibitors, digoxin (also called digitalis), and beta-blockers. In mild cases, thiazide diuretics, such as hydrochlorothiazide at 25-50 mg/day or chlorothiazide at 250-500 mg/day, are useful. However, supplemental potassium chloride is generally needed, since chronic diuresis causes hypokalemis alkalosis. Moreover, thiazide diuretics usually are not effective in patients with advanced symptoms of Heart failure. Typical doses of ACE inhibitors include captopril at 25-50 mg/day and quinapril at 10 mg/day. Numerous side effects are possible, including decreased blood pressure, renal insufficiency, potassium retention, and coughing. A more indirect component of heart failure management includes the recognition and control of factors that may be causing increased cardiac demands or adversely affecting myocardial function (e.g., hypertension, anemia, excess salt intake, excess alcohol, arrhythmias, thyrotoxicosis, fever, increased ambient temperature, or pulmonary emboli) (Beers and Berkow, eds., The Merck Manual of Diagnosis and Therapy, 17th ed. (Whitehouse Station, N.J.: Merck Research Laboratories, 1999) 1688-91). In view of the foregoing, many of the current methods available for treating heart failure produce negative side-effects and/or treat heart failure only indirectly. Accordingly, there currently exists a need for new and better methods for improving the survival of patients with heart failure.

Myocardial infarction (irreversible damage to heart tissue, often due to heart attack) is a common life-threatening event that may cause sudden death or heart failure. The ventricular dysfunction that arises after myocardial infarction results, primarily, from a massive loss of cardiomyocytes and gradual replacement of damaged cardiomyocytes with fibrotic non-contractile (scar) tissue. In most cases, the loss of cardiomyocytes after myocardial infarction is irreversible. Indeed, it is widely accepted that the proliferative (and, therefore, the regenerative) potential of adult mammalian cardiomyocytes is quite limited (Rumyantsev and Carlson, Growth and Hyperplasia of Cardiac Muscle Cells (New York: Harwood Academic Publishers, 1991)), although this view has recently been challenged (Leri et al., Mol. Cell. Cardiol., 3:385-90, 2000; Kajstura et al., Am. J. Pathol., 156:813-19, 2000; Beltrami et al., N. Engl. J. Med., 344(23):1750-57, 2001).

Despite considerable advances in the diagnosis and treatment of heart disease, cardiac damage and dysfunction relating to myocardial infarction are still among the major cardiovascular disorders. Accordingly, it remains a major therapeutic challenge to find new effective approaches to improve cardiac function after myocardial infarction.

Beta-1 and beta-2 adrenergic receptors are expressed in many organs of the body including the heart, lungs, and vascular tissue. These receptors mediate the actions of adrenaline and noradrenaline, as well as various synthetic agonists. In the heart, these receptors regulate heart rate and pumping function; in the lungs, they regulate bronchial tone; and in the vasculature, they regulate vascular tone. Beta-1 receptors are instrumental in regulating heart rate, while beta-2 receptors play an important role in regulating smooth muscle function.

Beta adrenergic blocking drugs (beta-blockers or beta-antagonists) were introduced in the early 1960s. They are commonly used to treat hypertension, congestive heart failure, arrhythmias, and angina, and are frequently used to prevent heart attacks in high-risk patients. Beta blockers may also be given to patients who have suffered a heart attack, in order to lessen oxygen consumption by the damaged heart muscle, and prevent sudden death. Beta-blockers slow the nerve impulses traveling through heart tissue by blocking the effects of adrenaline on beta receptors. Beta blockers also block the impulses that cause arrhythmia. Beta-blockers can be non-selective or selective for either beta-1 or beta-2 receptors. For example, Metoprolol, a frequently used beta-blocker, is a selective adrenergic beta-1 blocker. Metoprolol inhibits the agonistic effect of catecholamines (compounds which are released during physical and mental stress) on the heart. Thus, metoprolol reduces the increase in heart rate, cardiac output, cardiac contractility, and blood pressure produced by an acute increase in catecholamines. Adrenergic beta antagonists include, but are not limited to acebutolol, alprenolol, amosulalol arotinolol, atenolol, befunolol, betaxolol, bevantolol, bisoprolol, bopindolol, bucindolol, bufetolol, bufuralol, bunitrolol, bupranolol, butidrine hydrochloride, butofilolol, carazolol, careolol, carvedilol, celiprolol, cetamolol, cloranolol, dilevalol, esmolol, indenolol, labetalol, landiolol, vevobunolol, mepinodolol, metipranolol, metoprolol, moprolol, nadolol, nadoxolol, nebivolol, nifenalol, nipradilol, oxprenolol, penbutolol, pindolol, practolol, pronethalol, proranolol, sotalol, sulfinalol, talinolol, tertatolol, tilisolol, timolol, toliprolol, and xibenolol.

Adrenergic beta agonists include, but are not limited to albuterol, bambuterol, bitolterol, carbuterol, clenbuterol, clorprenaline, denopamine, dioxethedrine, dopexamine, ephedrine, epinephrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, prenalterol, procaterol, protkylol, reproterol, rimiterol, ritodrine, salmeterol, soterenol, terbutaline, tretoquinol, tulobuterol, and xamoterol. Selective adrenergic beta-2 receptor agonists mimic the effects of adrenaline and noradrenaline, and, therefore, can function as vasodilators. Beta-2 receptor agonists are traditionally used to relieve bronchiospasm in the treatment of respiratory diseases, such as asthma or chronic obstructive pulmonary disease, and are particularly useful in the treatment of asthma symptoms caused by bronchial constriction, including chest tightness, coughing, and wheezing. Clenbuterol is a long-acting beta 2-adrenergic agonist used in the treatment of pulmonary disorders.

Congestive heart failure (“CHF”) is a progressive pathophysiologic condition where cardiac function is impaired to a degree that the heart is unable to generate output sufficient to meet the metabolic requirements of the tissues and organs of the body. After initial cardiac injury, in order to compensate for the shortfall in output, myocardial workload increases, as does overall heart mass and size. The resulting condition of cardiac hypertrophy eventually leads to further ventricular dysfunction and heart failure. This maladaptive process is called cardiac remodeling.

CHF affects 4.7 million patients in the United States and is responsible for approximately one million hospitalizations and 300,000 deaths annually (americanheart.org/statistics). The total annual costs associated with this disorder have been estimated to exceed $22 billion (O'Connel J B, Bristow M R. Economic impact of heart failure in the United States: Time for a different approach. Heart Lung Transplant 1993; S107-S112.). When the disease enters its terminal phase, the only cure is heart transplantation. It is estimated that 15,000 patients would benefit from such a procedure. Unfortunately, due to a shortage of donor hearts, only 2,000 heart transplants are performed in the United States annually. (http://www.unos.org/data/). A need therefore exists for effective, non-transplant treatment for CHF.

Symptoms of CHF include fatigue, dyspnea and fluid retention in the lungs and extremities. Patients with CHF have reduced exercise capacity, also referred to as “exercise intolerance.” As CHF progressively becomes more severe, patients are unable to perform basic activities of daily life. Improving exercise capacity, and concomitantly quality of life, is therefore a primary goal in the management of CHF. The methods of treatment that are the subject of the present invention meet this need.

Reduced cardiac output has long been viewed as the cause of exercise intolerance. Prior art medical strategies for treating CHF have therefore focused on pharmacotherapy to improve hemodynamic function by lowering blood pressure (e.g., with vasodilators), decreasing fluid buildup (e.g., through use of diuretics) and increasing stroke volume (e.g., with digitalis preparations).

A variant on this hemodynamic-focused paradigm has been suggested. In a review article, Clark et al. (Clark A L, Poole-Wilson P A, Coats A J S. Exercise limitation in chronic heart failure: The central role of the periphery. J Am Col Cardiol 1996; 28:1092-1102) state that hemodynamic function is poorly related to exercise capacity and symptoms in CHF; instead, they state that abnormalities in the peripheral skeletal musculature are an important determinant of shortness of breath, fatigue and exercise limitation in CHF. Patients with CHF have been shown to have decreased muscle mass. However, this alone does not explain the marked abnormalities in skeletal muscle strength and exercise performance. Several human studies have reported abnormal biochemical and histological features in skeletal muscle biopsies of patients with CHF (Dunnigan A, Staley N A, Smith S A et al. Cardiac and skeletal muscle abnormalities in cardiomyopathy: comparison of patients with ventricular tachycardiac or congestive heart failure. J Am Col Cardiol 1987; 10:608-18; Lipkin D, Jones D, Round J, Poole-Wilson P. Abnormalities of skeletal muscle in patients with chronic heart failure. Int J Cardiology 1988; 18:187-95; Mancini D M, Coyle E, Coggan A, et al. Contribution of intrinsic skeletal muscle changes to ³¹P NMR skeletal muscle abnormalities in patients with chronic heart failure. Circulation 1989; 80:1338-46; Sullivan M J, Green H J, Cobb F R. Skeletal muscle biochemistry and histology in ambulatory patients with long-term heart failure. Circulation 1990; 81:518-27). Other human studies have shown abnormal skeletal muscle metabolism with rapid depletion of energy stores in CHF (Wiener D H, Fink L I, Maris J, et al. Skeletal muscle metabolism in patients with congestive heart failure: role of reduced muscle flow. Circulation 1986; 73:1127-36; Massie B M, Conway M, Yonge R, et al. ³¹P nuclear magnetic resonance evidence of abnormal skeletal muscle metabolism in patients with congestive heart failure. Am J Cardiol 1987; 60:309-15).

Clark et al. stated that pharmacological treatment focused on skeletal muscle abnormalities (for example, muscle-bulking agents, such as anabolic steroids and b-2 adrenoreceptor agonists) might improve patient quality of life. To date, no therapies are in use that target the skeletal muscle dysfunction in CHF.

While beta-2 adrenoreceptor agonists are primarily used therapeutically as bronchodilators in the treatment of asthma and chronic obstructive bronchitis, these agents are also reported in the medical and scientific literature as having, to varying degrees, anabolic effects on skeletal muscle. Of the beta-2 adrenoreceptor agonists, clenbuterol is known to have the most potent anabolic effect on skeletal muscle (Carter W J, Lynch M E. Comparison of the effects of salbutamol and clenbuterol on skeletal muscle mass and carcass composition in senescent rats. Metabolism 1994; 43:1119-1125). This anabolic effect may be related to clenbuterol's prolonged elevated serum levels. Oral clenbuterol has been used extensively by athletes to enhance muscle size and strength (Muscling in on clenbuterol. Lancet 1992; 340:403; Beckett A H. Clenbuterol and sport (letter). Lancet 1992; 340:1165; Clenbuterol: a medal in tablet form? (letter). Br J Sp Med 1993; 27:141).

Researchers in England have investigated possible use of beta-2 adrenoreceptor agonists to improve skeletal muscle functional capacity in humans. Two published studies involved participants in good cardiovascular health. In the first, Martineau et al. reported that twice-daily administration of sustained-release salbutamol (Volmax, 8 mg) increased voluntary muscle strength in healthy men (Martineau L, Horan M, Rothwell N J, Little R A Salbutamol a β₂-adrenoreceptor agonist, increases skeletal muscle strength in young men. Clin Sci 1992; 83:615-621). Maltin et al. reported that post-operative administration of clenbuterol to males with medial meniscus injury resulted in more rapid rehabilitation of strength in knee extensor muscles and concluded that beta-adrenoreceptor agonists have therapeutic potential in ameliorating muscle-wasting conditions in man (Maltin C A, Delday M I, Watson J S, Hyas S D, Nevison I M, Ritchie I K, Gibson P H. Clenbuterol, a beta-adrenoreceptor agonist, increases relative muscle strength in orthopaedic patients. Clin Sci 1993; 84:651-654). Use of clenbuterol, alone and in combination with beta-blockers, was later stated in three US patents, U.S. Pat. Nos. 5,530,029, 5,541,188 and 5,552,442, each issued to Maltin. These patents teach the use of clenbuterol as a treatment for muscle wasting conditions related to primary muscular disease, neuromuscular abnormalities, generalized catabolic disease states, such as cancer and acquired immune deficiency syndrome. The Maltin patents neither disclose nor suggest the use of clenbuterol for the skeletal muscle abnormalities of CHF.

The first and only study to date utilizing a beta-2 adrenoreceptor agonist to improve skeletal muscle function in CHF was reported by Harrington et al. (Harrington D, Chua T P, Coats A J S. The effect of salbutamol on skeletal muscle in chronic heart failure. Int J Cardiol 2000; 73:257-265). After three weeks of treatment with salbutamol (8 mg b.i.d.), there was no change in quadriceps bulk, muscle strength or fatigue and no change in exercise capacity or symptom assessment scores (Minnesota Living with Heart Failure Questionnaire). Accordingly, the investigators, themselves, described this as “essentially a negative study.”

Harrington et al. hypothesized that a more potent anabolic agent, such as clenbuterol, might have a role in the treatment of chronic heart failure myopathy. Persons of ordinary skill in cardiology would be quick to dismiss Harrington's suggestion for reasons of risk-benefit, particularly in light of the negative study results with salbutamol, the risk of mortality from arrhythmia when patients with CHF are treated with a beta-adrenergic agent, and data associating clenbuterol use with significant myocyte necrosis. More particularly, the risk of sudden cardiac death from arrhythmia in the overall adult population is 0.1-0.2 percent per year. In patients with CHF with reduced ejection fraction, the risk of sudden death from arrhythmia is 100 times greater (Myerburg R J. Kessler K M. Castellanos A. Structure, function, and time-dependence of risk. Circulation 1992. 85(1 Suppl):I2-10). Consequently, the administration of any pharmacological agent that might increase the risk of arrhythmia in a heart failure patient is avoided. Clenbuterol, like all b-2 adrenergic agents, has some b-1 cardiac activity and is therefore potentially arrhythmogenic in patients with abnormal cardiac function. Additionally, after demonstrating significant myocyte necrosis in both heart and skeletal muscle in rats treated with clenbuterol, Burniston et al. concluded, “clenbuterol may be damaging to long health” (Burniston J, N G Y, Clark W. Myotoxic effects of clenbuterol in the heart and soleus muscle. J App Physiol 2002; 93:1824-1832).

No investigators have studied clenbuterol for the treatment of skeletal myopathy in stable patients with CHF. However, Yacoub et al., have studied the effect of very high doses of clenbuterol on cardiac muscle in patients with advanced CHF supported with a left ventricular assist device (“LVAD”) (Yacoub M. A novel strategy to maximize the efficacy of left ventricular assist devices as a bridge to recovery. European Heart Jounral 2001; 22:534-540; Yacoub M, Tansley P, Birks E, Hipkin M, Hardy J, Bowles C, Banner N, Khaghani A. Interim results of left ventricular assist device combination therapy for inducing clinical and hemodynamic recovery of end stage dilated cardiomyopathy. Circulation 2002; 106(19):II-606 (Abstr); Hon J, Yacoub M. Bridge to Recovery with the Use of Left Ventricular Assist Device and Clenbuterol. Ann Thorac Surg 2003; 75:S36-41).

Patients who are critically ill with end stage heart failure sometimes require bridging mechanical support until a heart transplant becomes available. The LVAD is an implantable mechanical heart pump that unloads the left side of the heart and restores systemic blood flow. In the Yacoub study, patients with severe end stage non-ischemic cardiomyopathy supported with an LVAD were treated with high doses of clenbuterol with the goal of promoting recovery of cardiac function, in order to enable LVAD explantation without transplant. Supratherapeutic doses of clenbuterol—20 times greater than the recommended dose for treatment of asthma as well as 20 times the dose administered by Maltin et al. to post-operative orthopoedic patients—were administered to target the heart muscle and promote “physiological” myocyte hypertrophy.

Clenbuterol was given in combination with a standard heart failure medical regimen including a beta-1 selective blockade, ACE inhibitor, angiotensin-1 receptor antagonists and spironolactone. The theory underlying this combination therapy was that by first reducing hemodynamic load with the LVAD, “pathological” cardiac hypertrophy could be reversed and then replaced with “physiological” hypertrophy of cardiac muscle induced by clenbuterol.

The majority of patients in the Yacoub study demonstrated marked improvement in cardiac function sufficient to tolerate LVAD explantation without the need for cardiac transplantation. These preliminary results suggest a use for clenbuterol in a very limited subset of CHF patients—those with non-ischemic cardiomyopathy supported with an LVAD—and does not teach or support the use of clenbuterol in the non-LVAD, CHF population. The Yacoub study participants were protected from the consequences of arrhythmia by the LVAD, which in the event of circulatory collapse from arrhythmia, would mechanically support the circulation.

Clenbuterol hydrochloride is approved for use in the treatment of bronchial asthma and chronic obstructive bronchitis in Europe where it is manufactured by various manufacturers, including Boehringer Ingelheim, which sells the drug under the brand name Spiropent® in both tablet and liquid forms. Clenbuterol hydrochloride is not currently approved for any human use by the Food and Drug Administration in the United States, but has been approved for clinical study by applicant under an Investigational New Drug application.

According to the March 2000 package insert, Spiropent® is contraindicated for use in patients with tachycardic arrhythmia. There is also a caution for patients with severe coronary artery disease, who constitute approximately half of the heart failure population. The September 1995 Spiropent® Basic Product Information further advises that Spiropent® should be used only after careful risk-benefit assessment in patients with severe organic heart or vascular disorders. (Most patients with symptoms of CHF have severe organic heart disorder.) These warnings, combined with the general background knowledge in the cardiology community that patients suffering from heart failure are at increased for sudden death due to arrhythmia, would strongly teach those skilled in the art away from using clenbuterol in CHF patients not supported with an LVAD.

### SUMMARY OF THE INVENTION

The present invention provides compositions and methods for treating and preventing heart failure by administering to a patient a therapeutically effective amount of a beta-2 agonist. The present invention provides compositions and methods for treating and preventing heart failure resulting from ischemic and non-ischemic causes. In one embodiment of the invention, the beta-2 agonist is clenbuterol. In another embodiment, the beta-2 agonist is albuterol, formeoterol, levalbuterol, metaproterenol, pirbuterol, salmeterol, or terbutaline. In another embodiment, the method of the present invention treats or prevents heart failure by treating or preventing cardiac arrhythmia. In still another embodiment the method of the invention prevents or treats heart failure by treating or preventing tissue degeneration. In another embodiment, the present invention treats or prevents heart failure by treating or preventing heart tissue degeneration or reversing the effects of heart failure through normalization of calcium homeostasis. In an embodiment of the invention, the tissue degeneration can result from myocardial infarction. In an embodiment, the dosage of the beta-2 agonist is about 0.01 mg/kg/day to about 2.0 mg/kg/day. The beta-2 agonist clenbuterol can be administered in a dosage of about 5 mcg/day to 100 mg/day. Preferably, clenbuterol is administered in a dosage of bout 80 mcg/day to 1.5 mg/day.

The invention further provides compositions and methods for treating heart failure by administering to a patient a therapeutically effective amount of a beta-2 agonist in combination with a therapeutically effective amount of an adrenergic beta-1 antagonist. The beta-2 agonist and the beta-1 antagonist can be administered concurrently, sequentially or alternately. In a preferred embodiment, a synergistic therapeutic effect results from this combination therapy. The beta-2 agonist can be selected from the group consisting of clenbuterol, albuterol, formeoterol, levalbuterol, metaproterenol, pirbuterol, salmeterol, and terbutaline. In a preferred embodiment of the invention, the beta-2 agonist is clenbuterol. The beta-1 antagonist can be selected from the group consisting of acebutolol, atenolol, betaxolol, bisoprolol, esmolol, and metoprolol. In a preferred embodiment, the beta-1 antagonist is metoprolol. In an embodiment, the dosage of the beta-1 antagonist is about 15 mg/kg/day to about 300 mg/kg/day. The beta-1 antagonist metoprolol can be administered in a dosage of about 01 .mcg/day to 500 mg/day. Preferably, metoprolol is administered in a dosage of about 5 mg/day to 300 mg/day.

In another embodiment, the method of the present invention treats or prevents heart failure by treating or preventing cardiac arrhythmia. In still another embodiment the method of the invention prevents or treats heart failure by treating or preventing tissue degeneration. In one embodiment of the invention, heart failure can result from both ischemic and non-ischemic causes. In another embodiment of the invention, the tissue degeneration can result from myocardial infarction.

The invention further provides a method for preventing heart failure in a subject with a pre-heart failure condition, comprising administering to the subject a therapeutically effective amount of an adrenergic beta-2 agonist either alone or in combination with a therapeutically effective amount of an adrenergic beta-1 antagonist. When administered in combination, the beta-2 agonist and the beta-1 antagonist can be administered concurrently, sequentially or alternately. In a preferred embodiment a synergistic therapeutic effect results from this combination therapy. The beta-2 agonist can be selected from the group consisting of clenbuterol, albuterol, formeoterol, levalbuterol, metaproterenol, pirbuterol, salmeterol, and terbutaline. The dosages of the beta-2 agonist can be administered as previously described above. In a preferred embodiment of the invention, the beta-2 agonist is clenbuterol. The beta-1 antagonist can be selected from the group consisting of acebutolol, atenolol, betaxolol, bisoprolol, esmolol, and metoprolol. The dosages of the beta-1 antagonist can be administered as previously described above. In a preferred embodiment, the beta-1 antagonist is metoprolol.

In another embodiment, the method of the present invention prevents heart failure in a subject with a pre-heart failure condition by treating or preventing cardiac arrhythmia. In still another embodiment the method of the invention prevents heart failure in a subject with a pre-heart failure condition by treating or preventing tissue degeneration. In an embodiment of the invention, the tissue degeneration can result from myocardial infarction.

In another embodiment, the method of the present invention prevents heart failure in a subject post myocardial infarction by preventing cardiac arrhythmia. In still another embodiment the method of the invention prevents heart failure in a subject with a pre-heart failure condition by treating or preventing tissue degeneration. In an embodiment of the invention, the tissue degeneration can result from myocardial infarction.

The invention further provides a method for preventing heart failure in a patient status post myocardial infarction, comprising administering to the subject a therapeutically effective amount of an adrenergic beta-2 agonist either alone or in combination with a therapeutically effective amount of an adrenergic beta-1 antagonist. When administered in combination, the beta-2 agonist and the beta-1 antagonist can be administered concurrently, sequentially or alternately. In a preferred embodiment a synergistic therapeutic effect results from this combination therapy. The beta-2 agonist can be selected from the group consisting of clenbuterol, albuterol, formeoterol, levalbuterol, metaproterenol, pirbuterol, salmeterol, and terbutaline. The dosages of the beta-2 agonist can be administered as previously described above. In a preferred embodiment of the invention, the beta-2 agonist is clenbuterol. The beta-1 antagonist can be selected from the group consisting of acebutolol, atenolol, betaxolol, bisoprolol, esmolol, and metoprolol. The dosages of the beta-1 antagonist can be administered as previously described above. In a preferred embodiment, the beta-1 antagonist is metoprolol.

In another embodiment, the method of the present invention prevents heart failure in a patient status post myocardial infarction by treating or preventing cardiac arrhythmia. In still another embodiment the method of the invention prevents heart failure in a subject with a pre-heart failure condition by treating or preventing tissue degeneration. In an embodiment of the invention, the tissue degeneration can result from myocardial infarction.

The present invention additionally provides for a method of treating or preventing heart failure in a subject, comprising administering to the subject an amount of an adrenergic beta-2 agonist effective to treat or prevent the heart failure, in combination with an amount of an adrenergic beta-1 antagonist effective to reduce the toxicity of the adrenergic beta-2 agonist. In a preferred embodiment of the invention, the adrenergic beta-2 agonist is clenbuterol and the adrenergic beta-1 antagonist is metoprolol. In another embodiment of the invention the beta-2 agonist can be selected from the group consisting of clenbuterol, albuterol, formeoterol, levalbuterol, metaproterenol, pirbuterol, salmeterol, and terbutaline. The adrenergic beta-1 antagonist can be selected from the group consisting of acebutolol, atenolol, betaxolol, bisoprolol, esmolol, and metoprolol. The beta-2 agonist and the beta-1 antagonist can be administered concurrently, sequentially or alternately. Preferably, a synergistic therapeutic effect results from this combination therapy.

The present invention further provides a method for reversing damage to the heart following myocardial infarction using a combination of an adrenergic beta-1 antagonist and an adrenergic beta-2 agonist. In a preferred embodiment of the invention, the adrenergic beta-2 agonist is clenbuterol and the adrenergic beta-1 antagonist is metoprolol. In another embodiment of the invention the beta-2 agonist can be selected from the group consisting of clenbuterol, albuterol, formeoterol, levalbuterol, metaproterenol, pirbuterol, salmeterol, and terbutaline. The adrenergic beta-1 antagonist can be selected from the group consisting of acebutolol, atenolol, betaxolol, bisoprolol, esmolol, and metoprolol. The beta-2 agonist and the beta-1 antagonist can be administered concurrently, sequentially or alternately. Preferably, a synergistic therapeutic effect results from this combination therapy.

The present invention is additionally encompasses kits for use in treating or preventing heart failure and/or reversing damage to the heart following a heart attack comprising administering a combination of an adrenergic beta-1 antagonist and an adrenergic beta-2 agonist. In a preferred embodiment of the invention, the adrenergic beta-2 agonist is clenbuterol and the adrenergic beta-1 antagonist is metoprolol. In another embodiment of the invention the beta-2 agonist can be selected from the group consisting of clenbuterol, albuterol, formeoterol, levalbuterol, metaproterenol, pirbuterol, salmeterol, and terbutaline. The adrenergic beta-1 antagonist can be selected from the group consisting of acebutolol, atenolol, betaxolol, bisoprolol, esmolol, and metoprolol. The beta-2 agonist and the beta-1 antagonist can be administered concurrently, sequentially or alternately. Preferably, a synergistic therapeutic effect results from this combination therapy.

Additional aspects of the present invention will be apparent in view of the description which follows.

### BRIEF DESCRIPTION OF THE FIGURES

FIG. 1 is a bar graph representing relative infarct sizes, as expressed as a percentage of left ventricular circumference. While the LAD ligation animals had significantly larger infarct sizes than the Sham group, there were no differences in the infarct sizes among the LAD ligation groups.

FIG. 2 is a bar graph representing echocardiographic data, as expressed by fractional shortening (FS) and fractional area change (FAC). Each of the LAD ligation groups had a significantly lower FS and FAC than the Sham animals. There were no significant changes in FS or FAC between the baseline and endpoint parameters for any of the groups.

FIG. 3 is a bar graph representing Hemodynamic data after 12 weeks of follow-up. For left ventricular end-diastolic pressure (LVEDP), there were significantly higher diameters in the HF, Clen, and Clen+Meto groups versus the Sham group. However, metoprolol-treated rats had a lower LVEDP than the control HF group. For the maximum dP/dt, control HF and Clen rats had significantly lower values than Sham rats. There was no difference between the Meto or Clen+Meto group and the Sham group.

FIG. 4 is a graphical representation of ex vivo end-diastolic pressure-volume-relationship (EDPVR) tracings, after normalization of LV volumes for differences in heart weights. Clenbuterol-treated rats were shifted to the left vs. both control HF and Meto rats and were no different from Sham rats. In contrast, HF, Meto, and Clen+Meto rats had higher passive LV volumes than Sham rats.

FIG. 5A shows pictures representing photomicrographs representative of immunohistochemistry staining patterns for the apoptosis marker, TUNEL.

FIG. 5B shows pictures representing photomicrographs representative of immunohistochemistry staining patterns for the apoptosis marker, 8-oxoG.

FIG. 5C shows pictures representing photomicrographs representative of immunohistochemistry staining patterns for the apoptosis marker, OGG1.

FIG. 5D shows pictures representing photomicrographs representative of immunohistochemistry staining patterns for the apoptosis marker, MYH.

FIG. 6. Calcium-handling protein expression levels of the ryanodine receptor (RyR) and sarcoplasmic reticulum Ca²⁺-ATPase (SERCA₂ₐ), expressed relative to tubulin expression levels. The mean optical density units of RyR and SERCA^{2a} levels are decreased in the HF group, as compared to the Sham group. Clen-treated rats had significantly increased levels of RyR and SERCA₂ₐ vs. control HF rats. Representative autoradiographs are depicted for RyR and SERCA₂ₐ, with the corresponding tubulin blotting signals.

### DETAILED DESCRIPTION OF THE INVENTION

The following abbreviations are used in this example: LVAD, left ventricular assist device; HF, heart failure; LV, left ventricular; RyR, ryanodine receptor; SERCA2a, sarcoplasmic reticulum calcium-ATPase; LAD, left anterior descending artery; Clen, clenbuterol; Meto, metoprolol; Clen+Meto, clenbuterol and metoprolol; LVEDD, left ventricular end-diastolic diameter; LVEDA, left ventricular end-diastolic area; LVESD, left ventricular end-systolic diameter; LVESA, left ventricular end-systolic area; FS, fraction shortening; FAC, fractional area change; EDPVR, end-diastolic pressure-volume relationships; LVEDP, left ventricular end-diastolic pressure; LVSP, left ventricular systolic pressure; MAoP, mean aortic pressure; dP/dtmax, maximum left ventricular dP/dt; dP/dtmin, minimum left ventricular dP/dt; TUNEL, terminal deoxynucleotidyltransferase end labeling; ANOVA, analysis of variance; SD, standard deviation.

There are no therapies available that directly target the abnormal skeletal musculature in congestive heart failure. The present invention discloses novel methods for administering clenbuterol to patients not supported with an LVAD with all classes of congestive heart failure, from mild to severe, including acute and chronic heart failure syndromes, as well as patients with asymptomatic ventricular dysfunction and in patients after myocardial infarction. Clenbuterol will improve skeletal muscle function, exercise capacity, fatigue, quality of life, as well as cardiac function in patients with CHF.

For purposes of the present invention (except where context specifically requires an alternative construction), clenbuterol shall be understood to mean clenbuterol free base as well as pharmaceutical alternatives thereof containing the same therapeutic moiety (including pharmaceutically acceptable salts thereof, such as hydrochloride, hydrobromide etc., esters or complexes of the moiety) and includes the individual active optical isomers thereof (each alone or in non-racemic mixtures thereof), polymorphs and mixtures thereof.

According to the methods of treatment of the present invention, clenbuterol is administered to patients with CHF not supported with an LVAD. Clenbuterol may be administered by a variety of routes of administration, both immediate and extended release, including solid oral dosage forms (e.g., tablets, capsules), liquid oral dosage forms, intravenous or intramuscular or subcutaneous injection, topical administration or by inhalation. Clenbuterol supplements existing approved therapies for CHF including angiotensin converting enzyme inhibitors (ACE inhibitors), beta adrenoreceptor blocking agents, digoxin and spironolactone. As such co-therapy using clenbuterol and one or more of these agents in floating and fixed combinations are also suitable. Other medicinal combination therapy using clenbuterol with members of these classes and related classes of medicinal agents will be apparent to those of ordinary skill in the art.

The present invention also teaches methods for safely administering clenbuterol to improve skeletal muscle function in patients with CHF for whom administration of a beta adrenergic agonist, such as clenbuterol, would otherwise be contraindicated because of the risk of arrhythmia. As discussed above, patients with CHF and reduced ventricular function have a 100-fold increased risk of sudden death from arrhythmia. Clenbuterol possesses some beta-1 adrenergic activity and is potentially arryhthmogenic for patients with CHF.

According to the risk-stratified approach of the present invention, patients at highest risk for arrhythmia, such as those with ischemic cardiomyopathy or with a past history of arrhythmia, are administered clenbuterol in combination with a beta-1 selective blocker and after implantable Cardioverter-Defibrillator (“ICD”) implantation. The ICD is an electrical device used in patients at high risk for arrhythmia or in patients who have suffered an episode of significant arrhythmia such as ventricular tachycardia or sudden cardiac death. It detects serious arrhythmia and delivers therapy to restore normal rhythm. Patients previously taking a non-selective beta-blocking agent are switched to a beta-1 selective blocking agent. The use of a beta-1 selective blocking agent such as metoprolol or bisoprolol diminishes the cardiac effects of clenbuterol (through the beta-1 receptor) without diminishing its effect on skeletal muscle (through the beta-2 receptor).

Patients at lower risk for arrhythmia are administered clenbuterol in combination with a beta-1 selective blocker and without ICD. Patients considered at lowest risk for arrhythmia, such as those with mild ventricular dysfunction and in whom a beta-1 selective blocker was contraindicated (such as in obstructive airways disease and severe peripheral vascular disease), are administered clenbuterol without a beta-1 selective blocker or ICD. In the present invention, one embodiment is where the patient or subject is not supported by an LVAD.

Patients treated according to the method of treatment of the present invention are dosed over a broad range. Initially, patients receive a starting daily dose of about 40 mcg and are gradually up-titrated as tolerated to a maximal daily dose of about 4 mg. Up-titration occurs on a weekly basis in the absence of significant adverse effects.

The goal of the present invention is to improve skeletal muscle function, exercise capacity, quality of life and cardiac function in patients with CHF not supported with an LVAD. Objective measures of these parameters are well-known to persons of ordinary skill in the art and include the following. Skeletal muscle function is measured using standard tests of isometric muscle strength and fatigue and can be expressed as Maximal Strength (normalized for muscle cross-sectional area) and the Static Fatigue Index. Exercise capacity in patients with CHF is measured by cardiopulmonary exercise testing and is expressed as Peak Oxygen Consumption, Peak Work and Exercise Duration. Quality of life is measured using standard questionnaires such as the Minnesota Living with Heart Failure (MLHF) Questionnaire (Rector T S, Kubo S H, Cohn J N. Patients' self-assessment of their congestive heart failure. Part 2: Content, reliability and validity of a new measure, the Minnesota Living with Heart Failure questionnaire. Heart Failure 1987; October/November: 198-209.). Cardiac function is measured by echocardiography, or by cardiac nuclear scanning techniques (MUGA scan) and is expressed as Ejection Fraction.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the word “or” means any one member of a particular list and also includes any combination of members of that list.

The present invention encompasses methods for treating and preventing heart failure in a subject by administering to the subject a therapeutically effective amount of an adrenergic beta-2 agonist either alone or in combination with an adrenergic beta-1 antagonist.

The terms “congestive heart failure (CHF)”, “chronic heart failure”, “acute heart failure”, and “heart failure” are used interchangeably herein and refer to any condition characterized by abnormally low cardiac output in which the heart is unable to pump blood at an adequate rate or in adequate volume. When the heart is unable to adequately pump blood to the rest of the body, or when one or more of the heart valves becomes stenotic or otherwise incompetent, blood can “back up” into the lungs, causing the lungs to become congested with fluid. If this backward flow occurs over an extended period of time, heart failure can result. Typical symptoms of heart failure include shortness of breath (dyspnea), fatigue, weakness, difficulty breathing when lying flat, and swelling of the legs, ankles or abdomen (edema). Causes of heart failure are related to various disorders including coronary artery disease, systemic hypertension, cardiomyopathy or myocarditis, congenital heart disease, abnormal heart valves or valvular heart disease, severe lung disease, diabetes severe anemia hyperthyroidism, arrhythmia or dysrhythmia and myocardial infarction. The three cardinal signs of congestive heart failure are: cardiomegaly (enlarged heart), tachypnea (rapid breathing; occurs in the case of left side failure) and hepatomegaly (enlarged liver; occurs in the case of right side failure).

Treating heart failure, as used herein, refers to treating any one or more of the conditions underlying heart failure, including, without limitation, decreased cardiac contractility, abnormal diastolic compliance, reduced stroke volume, pulmonary congestion, and decreased cardiac output. As further used herein, “oxygen-wasting effects” include, without limitation, symptoms and signs of congestion due to increased ventricular filling pressures, and fatigue associated with low cardiac output. As used herein, preventing heart failure includes preventing the initiation of heart failure, delaying the initiation of heart failure, preventing the progression or advancement of heart failure, slowing the progression or advancement of heart failure, delaying the progression or advancement of heart failure, and reversing the progression of heart failure from an advanced to a less advanced stage.

In one embodiment of the invention, heart failure is treated in a subject in need of treatment by administering to the subject a therapeutically effective amount of an adrenergic beta-2 agonist effective to treat the heart failure. The subject is preferably a mammal (e.g., humans, domestic animals, and commercial animals, including cows, dogs, monkeys, mice, pigs, and rats), and is most preferably a human. The term “therapeutically effective amount,” or “effective amount” as used herein mean the quantity of the composition according to the invention which is necessary to prevent, cure, ameliorate or at least minimize the clinical impairment, symptoms or complications associated with heart failure in either a single or multiple dose. The amounts of adrenergic beta-2 agonist and beta-1 antagonist effective to treat heart failure will vary depending on the particular factors of each case, including the stage or severity of heart failure, the subject's weight, the subject's condition and the method of administration. The skilled artisan can readily determine these amounts.

Adrenergic beta-1 blockers (antagonists), such as metoprolol, have been used as essential therapies for heart-failure patients. However, prior to the present invention, adrenergic beta-2 agonists, such as clenbuterol, have traditionally been used to treat asthma. Clenbuterol, in particular, has been approved in the European Union for the treatment of asthma, and is often administered to athletes to improve performance capacity. The present invention establishes that adrenergic beta-2 agonists such as clenbuterol can also be used to prevent and treat heart-failure patients either alone or in combination with an adrenergic beta-1 antagonist such as metoprolol. This new therapy will provide a unique strategy to reverse the remodeling of the left ventricle during heart failure and after myocardial infarction (heart attack).

Metoprolol and clenbuterol target heart failure via different mechanisms: metoprolol blocks beta-1 receptors and corrects neurohormonal imbalance, while clenbuterol stimulates beta-2 receptors and rescues myocytes, is anti-apoptotic, and/or improves the function of calcium-handling proteins. It is believed that an adrenergic beta-1 blocker for use in the present invention will block the possible toxicity of high-dose adrenergic beta-2 agonist; the adrenergic beta-2 agonist is then expected to increase the mass of the heart (and skeletal muscle) physiologically, and rescue myocytes from apoptosis and necrosis. Thus, metoprolol and clenbuterol produce unexpected synergistic effects in the treatment of heart failure. Furthermore, clenbuterol, when used in combination with metoprolol, may be administered in amounts lower than would otherwise be expected.

As used herein, “adrenergic beta-2 agonist” refers to adrenergic beta-2 agonists and analogues and derivatives thereof, including, for example, natural or synthetic functional variants which have adrenergic beta-2 agonist biological activity, as well as fragments of an adrenergic beta-2 agonist having adrenergic beta-2 agonist biological activity. As further used herein, the term “adrenergic beta-2 agonist biological activity” refers to activity that mimics the effects of adrenaline and noradrenaline in a subject and which improves myocardial contractility in a patient having heart failure. Commonly known adrenergic beta-2 agonists include, but are not limited to, clenbuterol, albuterol, formeoterol, levalbuterol, metaproterenol, pirbuterol, salmeterol, and terbutaline.

As used herein, “adrenergic beta-1 antagonist” and adrenergic beta-1 blocker are used interchangeably and refer to adrenergic beta-1 antagonists and analogues and derivatives thereof, including, for example, natural or synthetic functional variants which have adrenergic beta-1 antagonist biological activity, as well as fragments of an adrenergic beta-1 antagonist having adrenergic beta-1 antagonist biological activity. As further used herein, the term “adrenergic beta-1 antagonist biological activity” refers to activity that blocks the effects of adrenaline on beta receptors. Commonly known adrenergic beta-1 antagonists include, but are not limited to, acebutolol, atenolol, betaxolol, bisoprolol, esmolol, and metoprolol.

Methods of preparing adrenergic beta-2 agonists such as clenbuterol and their analogues and derivatives are well known in the art. Clenbuterol, for example, is available from MP Biomedicals, Inc. 1263 S. Chillicothe Rd., Aurora, Ohio 44202. Clenbuterol is also commercially available under numerous brand names including Spiropent® (Boehinger Ingelheim), Broncodil® (Von Boch I), Broncoterol® (Quimedical PT), Cesbron® (Fidelis PT), and Clenbuter® (Biomedica Foscama). Similarly, methods of preparing adrenergic beta-1 antagonists such as metoprolol and their analogues and derivatives are well-known in the art. Metoprolol, in particular, is commercially available under the brand names Lopressor® (metoprolol tartate) manufactured by Novartis Pharmaceuticals Corporation, One Health Plaza, East Hanover, N.J. 07936-1080. Generic versions of Lopressor® are also available from Mylan Laboratories Inc., 1500 Corporate Drive, Suite 400, Canonsburg, Pa. 15317; and Watson Pharmaceuticals, Inc., 360 Mt. Kemble Ave. Morristown, N.J. 07962. Metoprolol is also commercially available under the brand name Toprol XL®, manufactured by Astra Zeneca, LP. Moreover, both beta-2 agonists and beta-1 antagonists may be synthesized in accordance with known organic chemistry procedures that are readily understood by those of skill in the art.

In a method of the present invention, an adrenergic beta-2 agonist is administered to a subject in combination with an adrenergic beta-1 agonist, such that a synergistic therapeutic effect is produced. A “synergistic therapeutic effect” refers to a greater-than-additive therapeutic effect which is produced by a combination of two therapeutic agents, and which exceeds that which would otherwise result from individual administration of either therapeutic agent alone. For instance, administration of clenbuterol in combination with metoprolol unexpectedly results in a synergistic therapeutic effect by providing greater efficacy than would result from use of either of the therapeutic agents alone. Clenbuterol enhances metoprolol's effects. Therefore, lower doses of one or both of the therapeutic agents may be used in treating heart failure, resulting in increased therapeutic efficacy and decreased side-effects.

In the method of the present invention, administration of an adrenergic beta-2 agonist “in combination with” an adrenergic beta-1 antagonist refers to co-administration of the two therapeutic agents. Co-administration may occur concurrently, sequentially, or alternately. Concurrent co-administration refers to administration of both the adrenergic beta-2 agonist and the adrenergic beta-1 antagonist at essentially the same time. For concurrent co-administration, the courses of treatment with the adrenergic beta-2 agonist and with the adrenergic beta-1 antagonist may be run simultaneously. For example, a single, combined formulation, containing both an amount of an adrenergic beta-2 agonist and an amount of an adrenergic beta-1 antagonist in physical association with one another, may be administered to the subject. The single, combined formulation may consist of an oral formulation, containing amounts of both the beta-2 agonist and the beta-1 antagonist, which may be orally administered to the subject, or a liquid mixture, containing amounts of both the beta-2 agonist and the beta-1 antagonist, which may be injected into the subject.

It is also provided by the present invention that an amount of the adrenergic beta-2 agonist and an amount of the adrenergic beta-1 antagonist may be administered concurrently to a subject, in separate, individual formulations. Accordingly, the method of the present invention is not limited to concurrent co-administration of the adrenergic beta-2 agonist and the adrenergic beta-1 antagonist in physical association with one another.

In the method of the present invention, the adrenergic beta-2 agonist and the adrenergic beta-1 antagonist also may be co-administered to a subject in separate, individual formulations that are spaced out over a period of time, so as to obtain the maximum efficacy of the combination. Administration of each therapeutic agent may range in duration from a brief, rapid administration to a continuous perfusion. When spaced out over a period of time, co-administration of the adrenergic beta-2 agonist and the adrenergic beta-1 antagonist may be sequential or alternate. For sequential co-administration, one of the therapeutic agents is separately administered, followed by the other. For example, a full course of treatment with the adrenergic beta-2 agonist may be completed, and then may be followed by a full course of treatment with the adrenergic beta-1 antagonist. Alternatively, for sequential co-administration, a full course of treatment with the adrenergic beta-1 antagonist may be completed, then followed by a full course of treatment with the adrenergic beta-2 agonist. For alternate co-administration, partial courses of treatment with the adrenergic beta-2 agonist may be alternated with partial courses of treatment with the adrenergic beta-1 antagonist, until a full treatment of each therapeutic agent has been administered.

The therapeutic agents of the present invention (i.e., the adrenergic beta-2 agonist and the adrenergic beta-1 antagonist, either in separate, individual formulations, or in a single, combined formulation) may be administered to a human or animal subject by known procedures, including, but not limited to, oral administration, parenteral administration (e.g., intramuscular, intraperitoneal, intravascular, intravenous, or subcutaneous administration), and transdermal administration. Preferably, the therapeutic agents of the present invention are administered orally or intravenously.

For oral administration, the formulations of the adrenergic beta-2 agonist either alone or in combination with the adrenergic beta-1 antagonist may be presented as capsules, tablets, powders, granules, or as a suspension. The formulations may have conventional additives, such as lactose, mannitol, corn starch, or potato starch. The formulations also may be presented with binders, such as crystalline cellulose, cellulose derivatives, acacia, corn starch, or gelatins. Additionally, the formulations may be presented with disintegrators, such as corn starch, potato starch, or sodium carboxymethyl cellulose. The formulations also may be presented with dibasic calcium phosphate anhydrous or sodium starch glycolate. Finally, the formulations may be presented with lubricants, such as talc or magnesium stearate.

For parenteral administration, the formulations of the adrenergic beta-2 agonist either alone or in combination with the adrenergic beta-1 antagonist may be combined with a sterile aqueous solution which is preferably isotonic with the blood of the subject. Such formulations may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampules or vials. Moreover, the formulations may be delivered by any mode of injection, including, without limitation, epifascial, intracapsular, intracutaneous, intramuscular, intraorbital, intraperitoneal (particularly in the case of localized regional therapies), intraspinal, intrasternal, intravascular, intravenous, parenchymatous, or subcutaneous.

For transdermal administration, the formulations of the adrenergic beta-2 agonist and the adrenergic beta-1 antagonist (whether individual or combined) may be combined with skin penetration enhancers, such as propylene glycol, polyethylene glycol, isopropanol, ethanol, oleic acid, N-methylpyrrolidone, and the like, which increase the permeability of the skin to the therapeutic agent, and permit the therapeutic agent to penetrate through the skin and into the bloodstream. The therapeutic agent/enhancer compositions also may be further combined with a polymeric substance, such as ethylcellulose, hydroxypropyl cellulose, ethylene/vinylacetate, polyvinyl pyrrolidone, and the like, to provide the composition in gel form, which may be dissolved in a solvent such as methylene chloride, evaporated to the desired viscosity, and then applied to backing material to provide a patch.

The dose of the adrenergic beta-2 agonist and the adrenergic beta-1 antagonist of the present invention may also be released or delivered from an osmotic mini-pump. The release rate from an elementary osmotic mini-pump may be modulated with a microporous, fast-response gel disposed in the release orifice. An osmotic mini-pump would be useful for controlling release, or targeting delivery, of the therapeutic agents.

It is within the confines of the present invention that the formulations of the adrenergic beta-2 agonist either alone or in combination with the adrenergic beta-1 antagonist may be further associated with a pharmaceutically-acceptable carrier, thereby comprising a pharmaceutical composition. The pharmaceutically-acceptable carrier must be “acceptable” in the sense of being compatible with the other ingredients of the composition, and not deleterious to the recipient thereof. Examples of acceptable pharmaceutical carriers include, but are not limited to, carboxymethyl cellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage.

The formulations of the present invention may be prepared by methods well-known in the pharmaceutical art. For example, the active compound may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the therapeutic agents of the present invention (i.e., the adrenergic beta-2 agonist and the adrenergic beta-1 antagonist, and their analogues and derivatives, either in separate, individual formulations, or in a single, combined formulation) to a subject to treat heart failure. The therapeutic agents are provided in amounts that are effective to treat or prevent heart failure in the subject. These amounts may be readily determined by the skilled artisan.

In the synergistic combination of the present invention, the adrenergic beta-2 agonist and the adrenergic beta-1 antagonist may be combined in a single formulation, such that the amount of the adrenergic beta-2 agonist is in physical association with the amount of the adrenergic beta-1 antagonist. This single, combined formulation may consist of an oral formulation, containing amounts of both the adrenergic beta-2 agonist and the adrenergic beta-1 antagonist, which may be orally administered to the subject, or a liquid mixture, containing amounts of both the adrenergic beta-2 agonist and the adrenergic beta-1 antagonist, which may be injected into the subject.

Alternatively, in the synergistic combination of the present invention, a separate, individual formulation of the adrenergic beta-2 agonist may be combined with a separate, individual formulation of the adrenergic beta-1 antagonist. For example, an amount of the adrenergic beta-2 agonist may be packaged in a vial or unit dose, and an amount of the adrenergic beta-1 antagonist may be packaged in a separate vial or unit dose. A synergistic combination of the adrenergic beta-2 agonist and the adrenergic beta-1 antagonist then may be produced by mixing the contents of the separate vials or unit doses in vitro. Additionally, a synergistic combination of the adrenergic beta-2 agonist and the adrenergic beta-1 antagonist may be produced in vivo by co-administering to a subject the contents of the separate vials or unit doses, according to the methods described above. Accordingly, the synergistic combination of the present invention is not limited to a combination in which amounts of the adrenergic beta-2 agonist and the adrenergic beta-1 antagonist are in physical association with one another in a single formulation.

The synergistic combination of the present invention comprises an effective therapeutic amount of the adrenergic beta-2 agonist and an effective therapeutic amount of the adrenergic beta-1 antagonist. As used herein, an “therapeutically effective amount” of the adrenergic beta-2 agonist or the adrenergic beta-1 antagonist is an amount of the adrenergic beta-2 agonist or the adrenergic beta-1 antagonist that is effective to ameliorate or minimize the clinical impairment or symptoms of heart failure in a subject, in either a single or multiple dose. For example, the clinical impairment or symptoms of heart failure may be ameliorated or minimized by diminishing any pain or discomfort suffered by the subject; by extending the survival of the subject beyond that which would otherwise be expected in the absence of such treatment; or by inhibiting or preventing the progression of the heart failure, or by reversing the pathologic processes involved in heart failure.

The effective therapeutic amounts of the adrenergic beta-2 agonist and the adrenergic beta-1 antagonist will vary depending on the particular factors of each case, including the stage of the heart failure, the subject's weight, the severity of the subject's condition, and the method of administration. For example, the beta-2 agonist clenbuterol can be administered in a dosage of about 5 mcg/day to 100 mg/day. Preferably, clenbuterol is administered in a dosage of bout 80 mcg/day to 1.5 mg/day.

In a preferred embodiment, the dosage of beta-2 agonist is about 0.01 mg/kg/day to about 2.0 mg/kg/day. In one embodiment, albuterol can be administered in a dosage of about 10 mcg/day to 200 mg/day, and preferably, is administered in a dosage about 2 mg/day to 32 mg/day. Formoterol can be administered in a dosage of about 1 mcg/day to 200 mg/day, and preferably, is administered in a dosage of about 12 mcg/day to 2 mg/day. Levalbuterol can be administered in a dosage of about 1 mcg/day to 200 mg/day, and preferably, is administered in a dosage of about 100 mcg/day to 100 mg/day. Metaproterenol can be administered in a dosage of about 0.1 mcg/day to 200 mg/day, and preferably, is administered in a dosage of about 2 mcg/day to 2 mg/day. Pirbuterol can be administered in a dosage of about 0.1 mcg/day to 200 mg/day, and preferably, is administered in a dosage of about 2 mcg/day to 2 mg/day. Salmeterol can be administered in a dosage of about 1 mcg/day to about 200 mg/day, and preferably, is administered in a dosage of about 2 mcg/day to 2 mg/day. Terbutaline can be administered in a dosage of about 0.1 mcg/day to 200 mg/day, and preferably, is administered in a dosage of about 5 mg to 20 mg/day.

The beta-1 antagonist metoprolol can be administered in a dosage of about 01 .mcg/day to 300 mg/day. Preferably, metoprolol is administered in a dosage of about 5 mg/day to 200 mg/day. Acebutolol can be administered in a dosage of about 50 mg/day to 5000 mg/day, and preferably, is administered in a dosage of about 200 mg/day to 1200 mg/day. Atenolol can be administered in a dosage of about 1 mg/day to 500 mg/day, and preferably, is administered in a dosage of about 25 mg/day to 100 mg/day. Betaxolol can be administered in a dosage of about 1 mg/day to 100 mg/day, and preferably, is administered in a dosage of about 5 mg/day to 20 mg/day. Bisoprolol can be administered in a dosage of about 0.1 mg/day to 200 mg/day, and preferably, is administered in a dosage of about 1 mg/day to 20 mg/day. Esmolol can be administered in a dosage of about 150 mcg/day to 100 gm/day, and preferably, is administered in a dosage of about 500 mg/day to 30 gm/day. Metoprolol can be administered in a dosage of about 1 mg/day to 500 mg/day, and preferably, is administered in a dosage of about 5 mg/day to 300 mg/day, and preferably, the dosage of beta-2 agonist is about 0.01 mg/kg/day to about 2.0 mg/kg/day and the corresponding dosage of beta-1 antagonist is 15 mg/kg/day to about 300 mg/kg/day.

The appropriate effective therapeutic amounts of the adrenergic beta-2 agonist and the adrenergic beta-1 antagonist within the listed ranges can be readily determined by the skilled artisan.

The present invention additionally encompasses methods for preventing heart failure in a subject with a pre-heart failure condition regardless of cause of the heart failure (e.g. whether from ischemic or non-ischemic causes) and regardless of the chronicity of the heart failure (e.g. acute or chronic), comprising administering to the subject a therapeutically effective amount of an adrenergic beta-2 agonist either alone or in combination with a therapeutically effective amount of a beta-1 antagonist. As used herein, “pre-heart failure condition” refers to a condition prior to heart failure. The subject with a pre-heart failure condition has not been diagnosed as having heart failure, but nevertheless may exhibit some of the typical symptoms of heart failure and/or have a medical history likely to increase the subject's risk to developing heart failure.

The invention further provides methods for treating or preventing heart failure in a subject post myocardial infarction, comprising administering to the subject a therapeutically effective amount of an adrenergic beta-2 agonist either alone, or in combination with a therapeutically effective amount of a beta-1 antagonist. As used in the present invention, “myocardial infarction” refers to the medical term for heart attack. Myocardial infarction occurs when the blood supply to an area of the heart is interrupted because of narrowed or blocked blood vessels. This can cause permanent damage to the heart muscle. Common symptoms include substernal, crushing chest pain that may radiate to the jaw or arms. Chest pains may be associated with nausea, sweating and shortness of breath.

“Postmyocardial infarction syndrome”, as used herein, refers to the complications of myocardial infarction (heart attack) such as fever, chest pain, and pericarditis (inflammation of the sac surrounding the heart). Preinfarction syndrome refers to the onset of unstable angina (chest pain that leads to a heart attack).

As used herein, “heart tissue degeneration” means a condition of deterioration of heart tissue, wherein the heart tissue changes to a lower or less functionally-active form. As described above, heart tissue damage or degeneration may be caused by, or associated with, a variety of disorders, conditions, and factors, including, without limitation, chronic heart damage, chronic heart failure, acute heart damage, acute heart failure, injury and trauma, cardiotoxins, radiation, oxidative free radicals, decreased blood flow, and myocardial infarction. Preferably, the heart tissue degeneration of the present invention was caused by myocardial infarction or heart failure.

The present invention provides compositions and methods for treating and preventing heart failure resulting from both ischemic and non-ischemic causes. As used herein, ischemia (ischaemia) refers to an insufficient blood supply to any part of the body.

The invention also provides methods for treating or preventing heart failure in a patient status post myocardial infarction, comprising administering to the subject a therapeutically effective amount of an adrenergic beta-2 agonist in combination with a therapeutically effective amount of a beta-1 antagonist. These methods encompass, in particular, methods for reversing damage to the heart immediately following myocardial infarction using an adrenergic beta-1 blocker such as metoprolol in combination with an adrenergic beta-2 agonist such as clenbuterol. In an embodiment, the present invention treats or prevents heart failure by treating or preventing heart tissue degeneration or reversing the effects of heart failure through normalization of calcium homeostasis. As used herein, “status post myocardial infarction” refers to the condition of a subject closely following occurrence of myocardial infarction. As described above, the combination of a beta-2 agonist and a beta-1 antagonist can be administered concurrently, sequentially or alternately. In a preferred embodiment, the beta-2 agonist is clenbuterol and the beta-1 antagonist is metoprolol.

The following examples illustrate the present invention, and are set forth to aid in the understanding of the invention, and should not be construed to limit in any way the scope of the invention as defined in the claims which follow thereafter.

### EXAMPLE 1

### Patient with CHF Treated with Clenbuterol

A patient with New York Heart Association Class III CHF with an ICD, currently taking a heart failure medical regimen, including carvedilol (a non-selective beta-blocking agent), lisinopril (an ACE inhibitor), digoxin and spiranolactone, has shortness of breath on moderate exertion and a peak oxygen consumption of 14 ml/kg/min during cardiopulmonary exercising testing. After changing from carvedilol to an equivalent dose of metoprolol sustained-release (a selective beta adrenergic blocker), clenbuterol hydrochloride is initiated at a dose of 20 mcg b.i.d. and up-titrated after one week to 40 mcg b.i.d. After six weeks on this well-tolerated dose, cardiopulmonary exercise testing is repeated and demonstrates an increased peak oxygen consumption of 17 ml/kg/day.

### EXAMPLE 2

### Clenbuterol Improves Cardiac Function

This example studies the therapeutic effects of clenbuterol on chronic heart failure, and determined its underlining mechanisms in patients not supported by LVAD.

Chronic heart failure was induced by coronary artery ligation in rats, and was confirmed by echocardiography, three weeks post-surgery. Three groups of rats were studied: (1) Chronic heart failure without clenbuterol (n=7); (2) chronic heart failure+clenbuterol (2 mg/kg/day; n=5); and (3) rats after sham operation (n=11). After 8 weeks of oral clenbuterol therapy, echocardiography and direct hemodynamic monitoring were performed. Rat hearts were then harvested for ex vivo left ventricular pressure-volume relationship (LVPVR) tracings, histologic (trichrome) sections, and molecular assays. Western analysis for myocardial calcium-handling proteins (ryanodine receptors (RyR), SERCA2a, phospholamban, and calcium-sodium exchanger) was assessed by densitometry.

Rats with coronary artery ligation developed chronic heart failure, as compared with Sham rats, as was evidenced by decreased left ventricular (LV) pressure (94±2.8 mmHg vs. 114±3.3 mmHg), LVdP/dt (2,570±384 mmHg/s vs. 3,728±193 mmHg/s), and LV fractional area change (35±3% vs. 53±2%), and elevated LVEDP (27±5 mmHg vs. 12±3 mmHg) (all p<0.05). Clenbuterol-treated chronic heart failure rats had significantly improved hemodynamic parameters (LV pressure: 100±6 mmHg; LVEDP: 15±6 mmHg; LVdP/dt: 3,250±325 mmHg/s) and fractional area change (42±1%) (all p<0.05), as compared to chronic heart failure rats without clenbuterol. There was a left-shift in the LVPVR (a lower volume for a given LV pressure=15 mmHg: 1.02 mlat 15 mmHg vs. 1.11 mlat 15 mmHg, p<0.05) from clenbuterol-treated chronic heart failure rats vs. chronic heart failure rats alone. Histology confirmed comparable infarct sizes in these two groups. Protein levels of RyR (1.6±0.4 vs. 2.8±0.8, p<0.05) and SERCA2a (1.7±0.5 vs. 2.2±0.4, p<0.05) were decreased in chronic heart failure rats, and recovered (RyR: 2.2±0.7, SERCA2a: 2.0±0.2, p<0.05) in clenbuterol-treated chronic heart failure rats.

These results indicate that clenbuterol improves cardiac function and left ventricular pressure-volume relationship (LVPVR) in subjects suffering from chronic heart failure. It is believed that the underlying mechanism may involve reverse remodeling via the normalization of calcium homeostasis.

### EXAMPLE 3

### Combination Therapy of Clenbuterol and Metoprolol

The benefits of a combined therapy of adrenergic beta-1 blockers and adrenergic beta-2 agonists for treating heart failure can be demonstrated in a rat ischemic heart failure model. Specifically, a combination of clenbuterol and nietoprolol can be used to treat rats suffering from ischemic heart failure.

Heart failure can be induced by coronary artery ligation in rats, and confirmed by echocardiography. Five groups of rats can then be studied: (1) heart failure without clenbuterol or metoprolol; (2) heart failure+clenbuterol (2 mg/kg/day) and metoprolol (200 mg/kg/day); (3) heart failure+metoprolol alone; (4) heart failure+clenbuterol alone; and (5) rats after sham operation. After approximately 8 weeks of oral therapy, echocardiography and direct hemodynamic monitoring can be performed. Rat hearts are harvested for ex vivo left ventricular pressure-volume relationship (LVPVR) tracings, histologic (trichrome) sections, and molecular assays. Western analysis for myocardial calcium-handling proteins (ryanodine receptors (RyR), SERCA2a, phospholamban, and calcium-sodium exchanger) can be assessed by densitometry.

It is expected that the results of this study will demonstrate the general benefits of a combined therapy of adrenergic beta-1 blockers and beta-2 agonists for heart failure. Rats with coronary artery ligation should develop heart failure, as compared with Sham rats, evidenced by decreased left ventricular (LV) pressure, LVdP/dt, and LV fractional area change, and elevated LVEDP. Accordingly, rats treated with a combination of clenbuterol and metoprolol will demonstrate the most improved hemodynamic parameters and fractional area change, as compared to heart failure rats treated with either clenbuterol alone or metoprolol alone, and heart failure rats receiving neither clenbuterol or metoprolol.

### EXAMPLE 4

### Treat and Prevent Heart Failure with Combination Therapy

The benefits of a combined therapy of adrenergic beta-1 blockers and adrenergic beta-2 agonists for treating rats post myocardial infarction can also be demonstrated in rats in a post myocardial infarction condition. Specifically, a combination of clenbuterol and metoprolol can be used to treat and prevent heart failure, and reverse damage to the heart in rats with post myocardial infarction condition.

Myocardial infarction can be induced in rats, and confirmed by echocardiography. Five groups of rats can then be studied: (1) post-myocardial infarction without clenbuterol or metoprolol; (2) post-myocardial infarction+clenbuterol (2 mg/kg/day) and metoprolol (200 mg/kg/day); (3) post-myocardial infarction+metoprolol alone; (4) post-myocardial infarction+clenbuterol alone; and (5) rats after sham induction of myocardial infarction. After approximately 8 weeks of oral therapy, echocardiography and direct hemodynamic monitoring can be performed. Rat hearts are harvested for ex vivo left ventricular pressure-volume relationship (LVPVR) tracings, histologic (trichrome) sections, and molecular assays. Western analysis for myocardial calcium-handling proteins (ryanodine receptors (RyR), SERCA2a, phospholamban, and calcium-sodium exchanger) can be assessed by densitometry.

It is expected that the results of this study will demonstrate the general benefits of a combined therapy of adrenergic beta-1 blockers and beta-2 agonists preventing heart failure in rats post-myocardial infarction. Myocardial infarction induced rats should develop heart failure or heart failure symptoms, as compared with Sham rats, evidenced by decreased left ventricular (LV) pressure, LVdP/dt, and LV fractional area change, and elevated LVEDP. Clenbuterol and metoprolol may have a significant sygergistic therapeutic effect. Accordingly, rats treated with a combination of clenbuterol and metoprolol will demonstrate the most improved hemodynamic parameters and fractional area change, as compared to post-myocardial infarction rats treated with either clenbuterol alone or metoprolol alone, and post-myocardial infarction rats receiving neither clenbuterol or metoprolol.

### EXAMPLE 5

### Clenbuterol Improves Calcium Homeostasis, Decreases Apoptosis, and Attenuates Diastolic Dysfunction in a Model of Ischemic Cardiomyopathy

This example shows the use of clenbuterol in an experimental model of ischemic heart failure. The benefit of the β2-adrenergic agonist, clenbuterol (Clen), in LVAD patients with dilated cardiomyopathy has been reported, but its effect on ischemic heart failure (HF) is unknown. This example investigates whether Clen improves cardiac function, induces reverse remodeling, decreases apoptosis, and has synergy with a β1-antagonist, metoprolol (Meto), in a model of ischemic HF. HF was induced by LAD ligation in rats and confirmed by echocardiography 3 weeks post-surgery. Rats were randomized to 5 groups: 1) HF without therapy; 2) HF+Clen; 3) HF+Meto; 4) HF+Clen+Meto; and 5) rats after sham surgery. After 9 weeks of therapy, echocardiographic, hemodynamic, and ex vivo end-diastolic pressure-volume relationship (EDPVR) measurements were obtained. Rats with LAD ligation developed HF as compared to Sham rats, with decreased fractional shortening and dP/dtmax and elevated LVEDP (all p<0.05). Clen-treated ° F. rats had increased weight gain and heart weights (p<0.05 vs HF rats). The Meto-treated group had a lower heart rate (p<0.01) and LVEDP (p<0.05) vs the HF group. Normalized EDPVR curves revealed a leftward shift in Clen rats vs Meto and HF. (p<0.05). Clen, Meto, and Clen+Meto groups all had significant decreases in TUNEL and 8-oxoG and increased MYH and OGG1 immunohistochemical signals (all p<0.05). Western blot levels of RyR and SERCA2a were decreased in HF rats vs Sham rats and improved in Clen-treated HF. rats. This example shows that Clen ameliorates calcium homeostasis, apoptosis, and EDPVR but does not have synergy with Meto in our model of ischemic HF.

Clenbuterol is a selective β2-adrenergic receptor agonist first used in the mid-1970s to treat asthma and is approved for use for this indication in Europe (Salorinne Y, Stenius B, Tukiainen P, Poppius H. Double-blind cross-over comparison of clenbuterol and salbutamol tablets in asthmatic out-patients. Eur J Clin Pharmacol. 1975; 8:189-95). The drug bears close structural similarity to albuterol, differing from the latter by the presence of chlorine atoms and an amine group in the benzene ring. These changes enhance its oral absorption and β-2 selectivity. Clenbuterol is recognized as a more potent β2-adrenergic agonist than albuterol (Id) and increases muscle bulk to a greater extent than other β2-agonists in animal models (Carter W J, Lynch M E. Effect of clenbuterol on recovery of muscle mass and carcass protein content following experimental hyperthyroidism in old rats. Comp Biochem Physiol Comp Physiol. 1994; 108:387-94) secondary to an anabolic effect that is mediated by β2-activation. As a result of its anabolic actions, oral clenbuterol has been used extensively by athletes to enhance muscle size and strength (id. and Muscling in on clenbuterol. Lancet. 1992; 340:403.)

Interest in clenbuterol has been recently sparked as a potential treatment for cardiac diseases, specifically in regard to improving cardiac mechanical properties. Petrou, et al found that clenbuterol administration led to hypertrophy of latissimus dorsi and cardiac muscle in rats (Petrou M, Wynne D G, Boheler K R, Yacoub M H. Clenbuterol induces hypertrophy of the latissimus dorsi muscle and heart in the rat with molecular and phenotypic changes. Circulation. 1995; 92:11483-9). Clenbuterol promotes cardiac hypertrophy in rats after proximal banding of the ascending aorta (Wong K, Boheler K R, Petrou M, Yacoub M H. Pharmacological modulation of pressure-overload cardiac hypertrophy: changes in ventricular function, extracellular matrix, and gene expression. Circulation. 1997; 96:2239-46.) Normal rat hearts treated with clenbuterol have also been shown to have elements of “physiologic” hypertrophy, with normal function, morphology, and calcium-handing protein mRNA levels (Wong K, Boheler K R, Bishop J, Petrou M, Yacoub M H. Clenbuterol induces cardiac hypertrophy with normal functional, morphological and molecular features. Cardiovasc Res. 1998; 37:115-22.). Finally, it was found that clenbuterol improves right ventricular systolic function after induction of right ventricular failure by banding of the pulmonary artery in sheep (Hon J K, Steendijk P, Petrou M, Wong K, Yacoub M H. Influence of clenbuterol treatment during six weeks of chronic right ventricular pressure overload as studied with pressure-volume analysis. J Thorac Cardiovasc Surg. 2001; 122:767-74).

An preliminary report describes the use of clenbuterol (in combination with ACE inhibition, β-1 selective blockade, and spironolactone) in patients with non-ischemic, dilated cardiomyopathy supported with a left ventricular assist device (LVAD) (Yacoub M H. A novel strategy to maximize the efficacy of left ventricular assist devices as a bridge to recovery. Eur Heart J. 2001; 22:534-540; Yacoub M H, Tansley P, Birks E J, et al. A novel combination therapy to reverse end-stage heart failure. Transplant Proc. 2001; 33:2762-4). Ten of 15 patients treated with clenbuterol in this study had significant cardiac improvement, allowing for LVAD explantation for recovery (Hon J K, Yacoub M H. Bridge to recovery with the use of left ventricular assist device and clenbuterol. Ann Thorac Surg. 2003; 75:S36-41). This series represents a rate (67%) of myocardial recovery that is more than double that of any previously reported study. Still, the effects of clenbuterol on ischemic cardiomyopathy have not been evaluated to date in experimental or clinical studies.

Recent reports show that the toxic effects of β-adrenergic stimulation is mediated primarily via β-1 receptors while β-2 receptor stimulation may be protective (Lefkowitz R J, Rockman H A, Koch W J. Catecholamines, cardiac beta-adrenergic receptors, and heart failure. Circulation. 2000; 101:1634-1637). Myocardial apoptosis has been implicated as a possible mechanism in the pathogenesis of HF progression (Kang P M, Izumo S. Apoptosis and heart failure: A critical review of the literature. Circ Res. 2000; 86: 1107-1113) and has been correlated with the degree of left ventricular (LV) remodeling (Abbate A, Biondi-Zoccai G G L, Bussani R, et al. Increased myocardial apoptosis in patients with unfavorable left ventricular remodeling and early symptomatic post-infarction heart failure. J Am Coll Cardiol. 2003; 41:753-760). Catecholamine-induced apoptosis (Zaugg M, Xu W, Lucchinetti E, et al. Beta-adrenergic receptor subtypes differentially affect apoptosis in adult rat ventricular myocytes. Circulation. 2000; 102:344-350) and apoptosis in post-infarction HF (Prabhu S D, Wang G, Luo J, et al. Beta-adrenergic receptor blockade modulates Bcl-Xs expression and reduces apoptosis in failing myocardium. J Mol Cell Cardiol. 2003; 35:483-393) has been shown to be primarily mediated via β-1 adrenergic receptors. This invention provides that the combination of a β-1 blocker, such as metoprolol, and β-2 agonist, such as clenbuterol, may be synergistic in their effects on HF.

This example utilizes a well-established model of ischemic, chronic HF in rats for this study. The goals are: (1) to examine the effects of clenbuterol on cardiac function and ventricular remodeling in ischemic cardiomyopathy both alone and in combination with metoprolol, and (2) to determine the underlining effects of clenbuterol on calcium homeostasis and apoptosis. For this latter objective, the effects of clenbuterol were evaluated on markers of apoptosis, DNA damage, and DNA repair in our chronic model of HF. In addition, the effects of clenbuterol on protein expression levels of the ryanodine receptor (RyR) and sarcoplasmic reticulum calcium-ATPase (SERCA₂ₐ) were studied.

### Methods

### Laboratory Animals

All studies were performed in compliance with the Guide for the Care and Use of Laboratory Animals (NRC 1996) and were approved by the Institutional Animal Care and Use Committee of Columbia University. Sprague-Dawley rats (Charles River Laboratories, Wilmington, Mass.) weighing 250 to 300 g were used for all experiments. Food and water were provided ad libitum, with rats housed in a light- and temperature-controlled room over the 12-week study period.

### Induction of Chronic Heart Failure

After the induction of general anesthesia with the use of intraperitoneal ketamine (75 mg/kg; Fort Dodge Animal Health, Fort Dodge, Iowa) and xylazine (5 mg/kg; Lloyd Laboratories, Shenandoah, Iowa), endotracheal intubation with a 16-guage angiocathether was performed. Rats were supported by a small animal ventilator (Harvard Apparatus, Holliston, Mass.). After performing a left thoracotomy, a sham operation (pericardiectomy only) or left anterior descending artery (LAD) ligation with a 7-0 polypropylene suture was performed. Subsequently, a chest tube (16-guage angiocathether) was placed in the left pleural space prior to closing the incision in three layers. Finally, the chest tube was used to aspirate the pleural cavity and removed after extubation.

A total of 69 rats were used in this study. LAD ligation surgery was performed in 60 rats, of which 37 (62%) survived three weeks post-surgery. Another 9 rats underwent a sham operation (group Sham), with 7 (78%) survivors. Three weeks post-operatively, echocardiography was performed to establish the baseline level of HF (as measured by fractional shortening). The LAD ligation rats were divided into 4 treatment groups matched for the degree of HF and were randomly assigned to one of the following therapies for 9 additional weeks: (1) rats receiving no therapy (group HF, n=9); (2) rats receiving high-dose clenbuterol at 1 mg/kg/day (group Clen, n=9); (3) rats receiving high-dose metoprolol at 200 mg/kg/day (group Meto, n=9); or (4) rats receiving concurrent high-dose clenbuterol at 1 mg/kg/day and high-dose metoprolol therapy at 200 mg/kg/day (group Clen+Meto, n=10).

Of the 44 surviving rats (37 post-LAD ligation and 7 Sham rats), 39 (89%) survived the 12 week follow-up period. There were no differences in the survival rates among the 5 groups. The final number of rats included: 7 Sham rats, 9 HF rats, 8 Clen rats, 7 Meto rats, and 8 Clen+Meto rats. The LAD ligation rats all had a myocardial infarction of sufficient size to induce HF, spanning≧20% of the LV circumference (as determined by trichrome staining).

### Oral Pharmacotherapy

Clenbuterol (ICN Biomedicals, Aurora, Ohio) was sonicated and subsequently dissolved in the drinking water. Metoprolol (Sigma-Aldrich, St. Louis, Mo.) was dissolved in the drinking water either alone or in combination with clenbuterol for the Clen+Meto group. The concentrations of clenbuterol and metoprolol were varied to keep the study drug dose delivered within a narrow therapeutic window based on daily water consumption. The average dosages of clenbuterol and metoprolol achieved were 1.1±0.1 mg/kg/day of clenbuterol (Clen group), 198±32 mg/kg/day of metoprolol (Meto group), and 1.1±0.1 mg/kg/day of clenbuterol and 232±20 mg/kg/day of metoprolol (Clen+Meto group). The treated drinking water was made fresh every 48-72 hours. Oral pharmacotherapy was continued for a total of 9 weeks.

### Echocardiography

Under mild isoflurane anesthesia, 2-D echo (Sonos-5500, Agilent Technologies, Palo Alto, Calif.) was performed 3 and 12 weeks post-surgery for baseline (pre-treatment) and post-treatment measures of cardiac function, respectively. All echocardiography was performed and analyzed by a single, experienced individual (I.H.) in a blinded fashion. For each echo, LV anteroposterior diameter and short-axis area at the papillary muscle level were measured to obtain the LV end-diastolic diameter (LVEDD) and area (LVEDA) and end-systolic diameter (LVESD) and area (LVESA). Fraction shortening (FS) was calculated as [(LVEDD−LVESD)/LVEDD×100%] and fractional area change (FAC) was calculated as [(LVEDA−LVESA)/LVEDA×100%].

### Hemodynamic Measurements

In the terminal experiments 12 weeks post-surgery, the rats were anesthetized with inhaled isoflurane mixed with oxygen. A 2-French Millar catheter (Millar Instruments, Houston, Tex.) was inserted into the right carotid artery and pressure measurements collected and saved as the catheter was advanced into the left ventricle. The heart was quickly arrested in diastole and excised. Hearts were subsequently weighed and used for the ex vivo determination of LV end-diastolic pressure-volume relationships (EDPVR). Using Chart 4 (version 4.2.4, ADInstruments, Colorado Springs, Colo.), left ventricular end-diastolic pressure (LVEDP), left ventricular systolic pressure (LVSP), mean aortic pressure (MAoP), heart rate, and maximum and minimum LV dP/dt (dP/dtₘₐₓ and dP/dtₘᵢₙ) were later obtained from stored hemodynamic recordings by a single individual (S.X.), who was blinded to animals' group assignments.

### Measurements of Body Weight and Heart Weight

Body weight was measured pre-surgery and 3 and 12 weeks post-surgery. Heart weight was measured immediately after the heart was excised. The ratio of body weight to heart weight was subsequently calculated.

### Determination of End-Diastolic Pressure-Volume Relationships

An 18-gauge angiocatheter was placed into the LV through the aortic valve and connected to a three-way stopcock. A fine hemostat was placed on the atrial side of the mitral annulus to seal the LV. After eliminating any air from the system, LV pressures were measured using a 5-French Millar micromanometer introduced through the angiocatheter. While recording LV pressure using an analog-to-digital conversion reading system (Chart 4, version 4.2.4; ADInstruments, Colorado Springs, Colo.), saline was infused into the LV cavity in 50 μL increments using a calibrated 1 mL syringe. The infused fluid was withdrawn and measured to ensure that no leakage had occurred. Data was utilized only if >95% of the injected volume was recovered. Using commercial software (Igor Pro, version 4.0.5.1; WaveMetrics, Lake Oswego, Oreg.), values of LV pressure (P) and volume (V) were fitted according to the equation:

P=βV^{α},

where β is the base constant and α is an index of ventricular stiffness, as previously described (Mirsky I. Assessment of passive elastic stiffness of cardiac muscle: mathetical concepts, physiologic and clinical considerations, directions of future research. Prog Cardiovasc Dis. 1976; 18:277-308). Averaged data were then used to construct the mean LVPVR tracings for each group, after normalizing LV volumes for differences in heart weight, as reported previously (Rabkin D G, Jia C X, Cabreriza S E, et al. A novel arresting solution for study of postmortem pressure—volume curves of the rat left ventricle. J Surg Res. 1998; 80:221-8; Amirhamzeh M M, Hsu D T, Cabreriza S E, Jia C X, Spotnitz H M. Myocardial edema: comparison of effects on filling volume and stiffness of the left ventricle in rats and pigs. Ann Thorac Surg. 1997; 63:1293-7) Analyses were performed by a single individual (S.K.), who was blinded to the group assignment. Comparisons between groups were made based on normalized LV volume measurements at LV pressures of 30 mmHg.

### Histological Analysis and Tissue Harvest

A short-axis section of the heart at the point of maximal infarction was fixed in 4% paraformaldehyde solution for 12 hours. Sections were then embedded in paraffin and 5 μm slices used for trichrome staining. The infarct size was determined as a percentage of the LV circumference under light microscopy by a single individual (S.X.) blinded to group assignment. The remaining heart tissue was flash-frozen in liquid nitrogen and stored at −80° C. for Western blot analysis.

### Apoptosis Studies

Histological sections (5 μm thick) were used to perform immunohistochemistry for: terminal deoxynucleotidyltransferase end labeling (TUNEL; apoptosis marker), 8-oxoG (DNA damage product), MYH (DNA mismatch repair enzyme), and OGG1 (DNA base excision repair enzyme). The percentage of myocytes with nuclear staining was quantified in a blinded manner. Because OGG1 is found both in the nucleus and cytoplasm, a scoring system was utilized to semi-quantitatively describe the staining observed: 0=no staining; 1=0 to 15% staining; 2=15 to 30% staining; 3=30 to 50% staining; and 4=>50% staining of the nucleus and cytoplasm.

### Western Blot Analysis

Five rat hearts from each group (HF, Clen, Meto, Clen+Meto, and Sham) were randomly selected and used for analysis of calcium-handling protein expression levels. Lysates of LV tissue were obtained with the use of a homogenizer (Brinkmann Instruments, Westbury, N.Y.). Approximately 150 mg of heart tissue was placed in a seven-fold volume of lysis buffer (20 mM/L Na-HEPES, 4 mM/L EGTA, 1 mM/L DTT, pH 7.4) in the presence of proteinase inhibitors (0.1 mM/L leupeptin and 0.3 mM/L PMSF). The protein concentration was determined using a protein assay kit (Bio-Rad Laboratories, Hercules, Calif.). Samples (50 μg) were denatured at 95° C. and size-fractionated using SDS-polyacrylamide gel electrophoresis (PAGE) under reducing conditions. SDS-PAGE was performed using 7.5% separating and 5% stacking gels for SERCA₂ₐ and 5% separating and 4% stacking gels for RyR. Electrophoresis was performed in a Miniprotean II cell (Bio-Rad Laboratories, Hercules, Calif.) followed by transfer of proteins (using 34 V overnight at 4° C.) onto nitrocellulose in a mini trans-blot transfer cell (Bio-Rad Laboratories, Hercules, Calif.) filled with transfer buffer (25 m/L Tris-HCl, pH 8.3, 192 mM/L glycine and 20% methanol).

Blots were blocked overnight at 4° C. in 5% nonfat milk diluted in TBS-T (20 mM/L Tris-HCl, pH 7.6 and 137 mM/L NaCl with 0.1% Tween-20). Blots were then incubated with primary antibody diluted in TBS-T (anti-SERCA₂ₐ: 1:1,000, ABR Affinity BioReagents, Golden, Colo.; anti-RyR: 1:2,500, gift from Dr. Andrew Marks' laboratory; anti-tubulin: 1:1,000, Sigma-Aldrich) for 1 hour at room temperature. After washing in TBS-T, blots were incubated in the presence of a horseradish, peroxidase-labeled secondary antibody (SERCA₂ₐ: anti-mouse IgG, Amersham Biosciences, Piscataway, N.J.; RyR: anti-rabbit IgG, Amersham Biosciences) diluted 1:4,000 for 40 minutes at room temperature. Blots were washed again with TBS-T and then developed using ECL reagent (Amersham Biosciences), followed by autoradiography.

Optical densities of protein level signals were quantified with the use of a laser scanning densitometer (Molecular Dynamics, Palo Alto, Calif.) in a blinded manner. SERCA₂ₐ and RyR protein levels were expressed relative to levels of tubulin.

### Statistical Analysis

All statistical analysis was performed using SPSS 11.5 software (SPSS, Chicago, Ill.). Comparisons between treatment groups for hemodynamic data were made using a 2-way analysis of variance (ANOVA), with the group and infarct size [categorized as a large (≦30% of LV circumference) or small infarct (>30% of LV circumference)] as fixed factors. Comparisons of paired echocardiographic and body weight data from pre-treatment and post-treatment timepoints were performed with the use of repeated measures ANOVA, with the group, infarct size, and timepoint as fixed factors. Tukey's ad hoc tests were used for all comparisons between groups. A p-value less than 0.05 was considered statistically significant. All data are expressed as a mean±the standard deviation (SD).

### Results

### Histological Analysis

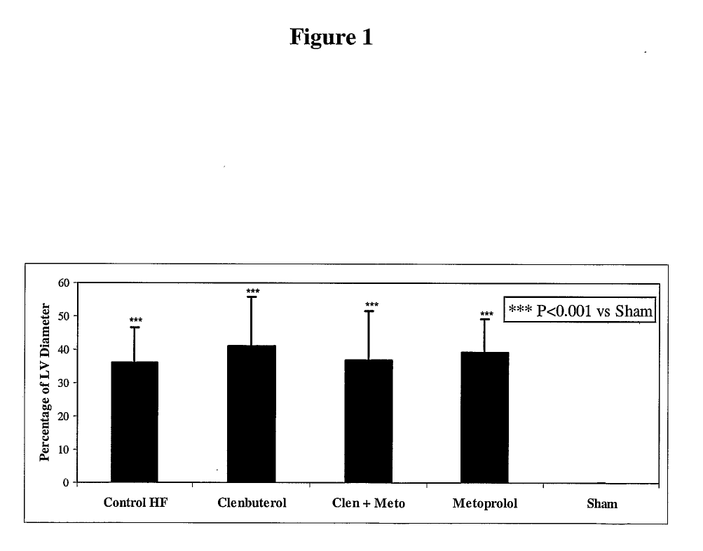

There were no significant differences in size of the LV infarct, as expressed as a percentage of the LV circumference, among the groups undergoing LAD ligation (see FIG. 1). Adequate LV infarctions were attained after all LAD ligations, with the range of infarct sizes spanning from 20 to 63% of the LV circumference (mean: 38.1±12.2%).

### Echocardiographic Data

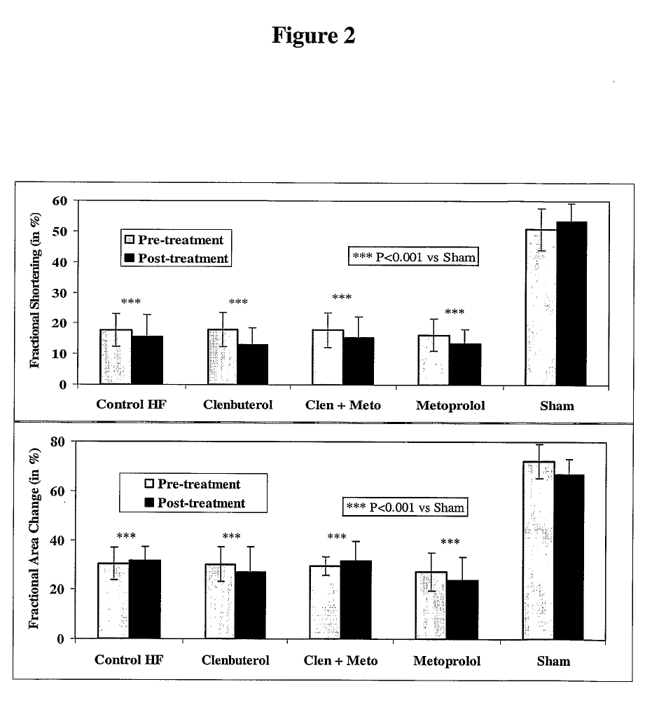

FIG. 2 depicts the short-axis echocardiographic data for the 5 groups at 3 and 12 weeks after surgery. At 3 weeks post-surgery, there was a significantly lower FS (17.4±5.22) and FAC (29.4±6.20) in the 4 LAD ligation groups, as compared to Sham rats (FS: 50.8±6.78; FAC: 72.3±6.89). There were no differences in the FS or FAC among the 4 LAD ligation groups prior to oral pharmacotherapy. After 9 weeks of therapy, there were again no differences in the FS or FAC among any of the groups.

### Body and Heart Weights

The body weight and heart weight data are shown in Table 1. The percentage change in the body weight was significantly higher in the Sham rats, as compared to all the LAD ligation groups. Treatment with clenbuterol alone and in combination with metoprolol, however, led to significantly higher increases in body weight, as compared to the control HF group. For heart weight, Sham rats had lower weights than all the LAD ligation rats. Clenbuterol-treated rats had significantly higher heart weights than both control HF and metoprolol-treated animals.

TABLE 1
Body and heart weight data.

| | HF | Clen | Meto | Clen + Meto | Sham |
|---|---|---|---|---|---|
| % Δ Weight | 33.8 ± 4.79*** | 49.9 ± 7.18** † | 32.3 ± 7.16*** | 55.3 ± 9.91* § † | 76.1 ± 19.3 |
| Heart Wt (gm) | 2.16 ± 0.33* | 2.64 ± 0.41*** § † | 2.17 ± 0.34* | 2.42 ± 0.32*** | 1.72 ± 0.09 |

Notes:
Values are expressed as the mean ± SD. % Δ weight represents the percentage change in pre-treatment to post-treatment weights.
*p < 0.05 vs Sham.
**p < 0.01 vs Sham.
***p < 0.001 vs Sham.
† p < 0.01 vs. HF.
§ p < 0.05 vs. Meto.
HF = heart failure, Clen = clenbuterol, Meto = metoprolol, Wt = weight.

### Hemodynamic Data

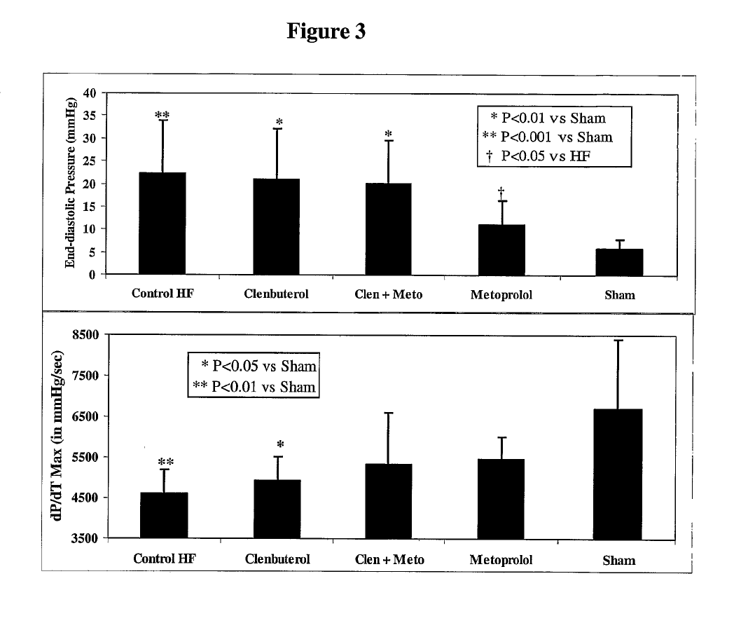

Table 2 depicts the direct hemodynamic data obtained in the study animals after 9 weeks of oral pharmacotherapy. Metoprolol-treated animals had a significantly lower heart rate than control HF rats. For LVEDP, while the control HF, Clen, and Clen+Meto group had a significantly higher LVEDP than Sham rats, the Meto rats were no different from Sham rats and had a lower LVEDP than HF rats (see FIG. 3). There were no differences in the systolic or mean LV or aortic pressures among the groups.

TABLE 2
Hemodynamic Data.

| | HF | Clen | Meto | Clen + Meto | Sham |
|---|---|---|---|---|---|
| HR (bpm) | 302 ± 19.1 | 281 ± 11.2 | 243 ± 19.4 ‡ | 275 ± 18 | 291 ± 52.2 |
| LVEDD (mmHg) | 22.4 ± 11.6*** | 21.1 ± 11.0** | 11.1 ± 5.29 † | 20.1 ± 9.53** | 5.92 ± 1.85 |
| LVSP (mmHg) | 113 ± 10.3 | 109 ± 10.7 | 107 ± 9.27 | 108 ± 11.1 | 116 ± 17.3 |
| MAP (mmHg) | 47.2 ± 8.85 | 44.2 ± 6.06 | 42.7 ± 5.38 | 43.4 ± 4.77 | 39.8 ± 5.56 |
| Maximum dP/dt | 4609 ± 583** | 4933 ± 596* | 5462 ± 541 | 5327 ± 1270 | 6700 ± 1706 |
| (mmHg/s) | | | | | |
| Minimum dP/dt | −3854 ± 563*** | 3901 ± 694** | −4032 ± 830** | −4117 ± 1035** | −6458 ± 1799 |
| (mmHg/s) | | | | | |

Notes:
Values are expressed as the mean ± SD.
*p < 0.05 vs Sham.
**p < 0.01 vs Sham.
***p < 0.001 vs Sham.
† p < 0.05 vs HF.
‡ p < 0.01 vs. HF.
HF = heart failure, Clen = clenbuterol, Meto = metoprolol, HR = heart rate, LVEDD = left ventricular end-diastolic diameter, LVSP = left ventricular systolic pressure, MAP = mean arterial pressure.

### End-Diastolic Pressure-Volume Relationship Tracings

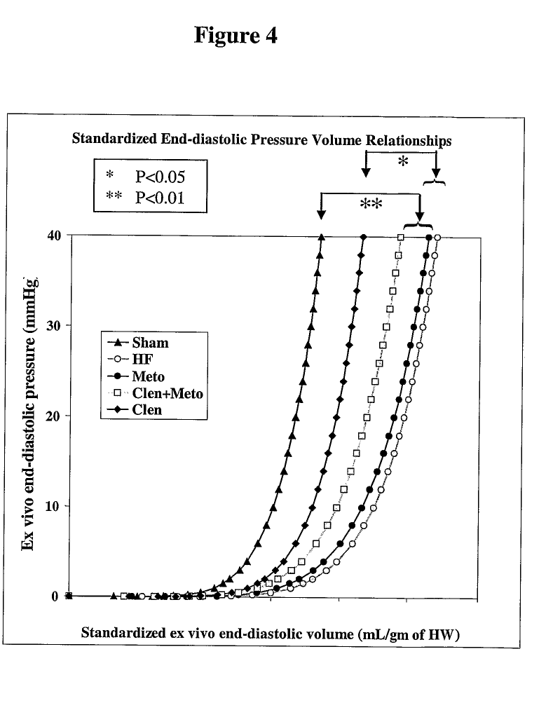

The ex vivo, passive EDPVR curves obtained are shown in FIG. 4, after normalization for differences in heart weight. There was a rightward shift for HF, Meto, and Clen+Meto versus Sham rats. In contrast, clenbuterol-treated rats (Volume at 30 mmHg: 0.42 mL/gm of heart weight) had lower passive LV volumes than either Meto or HF rats (Volume at 30 mmHg: 0.51 and 0.50 mL/gm of heart weight, respectively) and were no different from Sham rats (0.36 mL/gm of heart weight).

### Apoptosis Studies

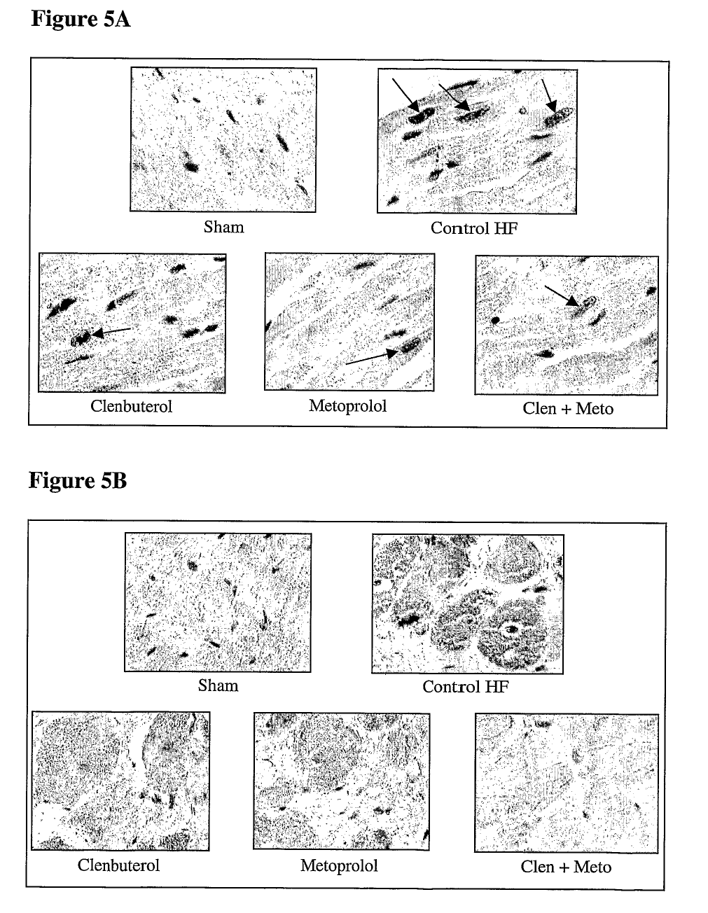

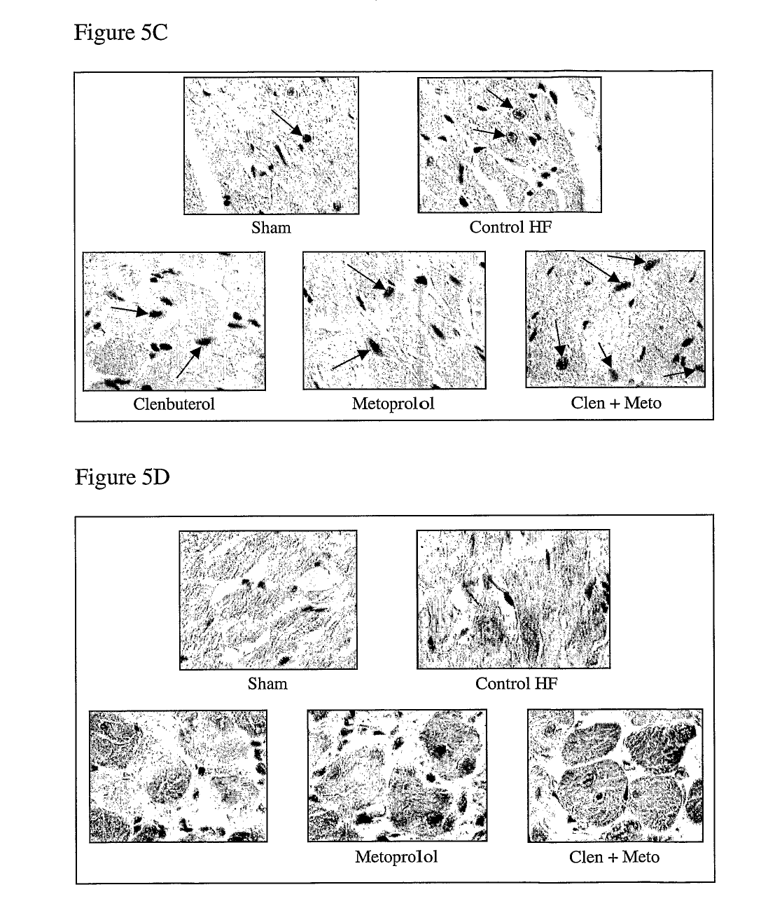

The quantitative immunohistochemistry staining of apoptosis, DNA damage, and DNA repair markers is shown in Table 3 and representative images are shown in FIG. 5. The staining of TUNEL was significantly increased in all the LAD ligation groups versus the Sham rats, indicating that there was increased apoptosis even 12 weeks after surgery in these animals. Clen and Meto treatment both alone and in combination led to decreased levels of TUNEL staining versus the HF group. Similarly, for 8-oxoG staining, the control HF, Clen, and Meto groups had increased levels of this DNA damage marker than Sham rats, though Clen, Meto, and Clen+Meto animals had decreased levels versus the HF rats. Clen+Meto treatment had an additive effect over Clen or Meto therapy alone, as seen by significantly decreased levels of DNA damage with the Clen+Meto group over Clen or Meto alone.

TABLE 3
Quantitative immunohistochemistry staining of
apoptosis, DNA damage, and DNA repair markers.

| | HF | Clen | Meto | Clen + Meto | Sham |
|---|---|---|---|---|---|
| TUNEL (%) | 21 ± 4* | 11 ± 3* † | 12 ± 4* † | 8.2 ± 2* † | 0.5 ± 0.1 |
| 8-oxoG (%) | 18 ± 4* | 10 ± 2* † | 12 ± 3* † | 5 ± 2 † ‡ | 0.5 ± 0.1 |
| MYH (score) § | 1.4 ± 0.5* | 2.8 ± 0.5* † | 2.6 ± 0.4* † | 3.2 ± 0.6* † | 0.6 ± 0.2 |
| OGG1 (%) | 0.5 ± 0.1* | 8.2 ± 2.2* † | 6.4 ± 1.5* † | 13 ± 4* † ‡ | 0.2 ± 0.1 |

Notes:
Values are expressed as the mean ± SD.
*p < 0.05 vs Sham.
† p < 0.05 vs HF.
‡ p < 0.05 vs Clen and vs Meto.
§ scoring system for MYH nuclear and cytoplasmic staining: 0 = 0%; 1 = 0-15%; 2 = 15-30%; 3 = 30-50%; and 4 = >50% positive.
Clen = clenbuterol, M = metoprolol, HF = heart failure.

For the MYH and OGG1 (markers of DNA repair), there were increased levels of both markers versus the Sham group for all the LAD ligation groups. Clen and Meto treatment led to improvements both alone and in combination versus the control HF group. Interestingly, Clen+Meto treatment led to additive improvement in the OGG1 staining pattern over either Clen or Meto therapy alone. In this study, there was no synergy noted in the effects of Clen+Meto therapy in any of the apoptosis, DNA damage, or DNA repair marker staining patterns.

### Western Blot Analysis

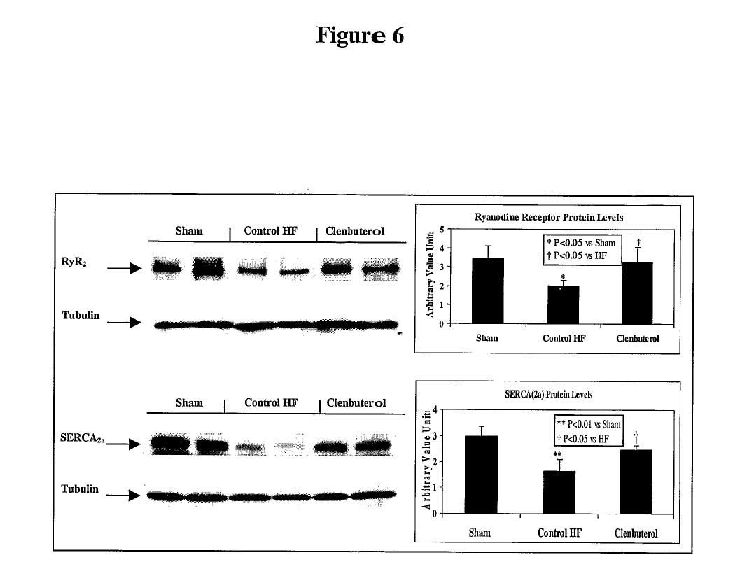

FIG. 6 depicts the calcium-handling protein expression levels for the RyR and SERCA₂ₐ. The HF rats had significantly decreased levels of both RyR and SERCA₂ₐ versus Sham rats. These levels were significantly improved with the Clen group versus the HF group, and these levels were not different from those of the Sham group.

### Discussion

This is the first study to date to evaluate the effects of the β-2 adrenergic agonist, clenbuterol, in ischemic cardiomyopathy. In this model of chronic, ischemic HF, clenbuterol treatment led to clear improvements in calcium homeostasis, diastolic function, myocardial apoptosis and DNA repair. In this study, no evidence was found of synergy in the use of this β-2 agonist and a selective β-1 antagonist, metoprolol.

Based on our echocardiographic and histological data, we were able to achieve a significant degree of HF in the experimental model. The size of the LV infarction attained was reproducible and uniform across the treatment arms. In addition, the FS and FAC on echocardiography demonstrated a significant decrease in LV systolic function in our chronic model of HF. Finally, repeat echocardiographic and direct hemodynamic data at 3 months demonstrated both systolic and diastolic dysfunction in the control HF group verus Sham rats.

High-dose clenbuterol treatment led to significant increases in heart weight and body weight over 9 weeks of oral pharmacotherapy. In contrast, high-dose metoprolol treatment led to both decreased heart rate and LVEDD with oral therapy. Although serum drug levels were not monitored in our experiment, these findings are strong evidence of effective delivery of each therapy. In addition, the improvements demonstrated with metoprolol therapy are similar to prior published reports in similar experimental rat post-infarction models, which have shown decreased heart rate and LVEDD (Prabhu S D, Chandrasekar B, Murray D R, Freeman G L. Beta-adrenergic blockade in developing heart failure: Effects on myocardial inflammatory cytokines, nitric oxide, and remodeling. Circulation. 2000; 101:2103-2109; Yang Y, Tang Y, Ruan Y, et al. Comparison of metoprolol with low, middle, and high doses of carvedilol in prevention of postinfarction left ventricular remodeling in rats. Jpn Heart J. 2003; 44:979-988) but no change in dP/dtₘₐₓ (id) with metoprolol treatment.

Clenbuterol treatment led to improved diastolic function in the Clen group, as seen by a rightward shift in the normalized EDPVR. HF hearts exhibited substantially enlarged LV volumes relative to non-infarcted Sham hearts, with a rightward shift of the pressure-volume curve. Clenbuterol therapy, however, caused a reduction in ventricular cavity dilation, shifting EDPVR curves leftward. This suggests that clenbuterol attenuated deleterious post-infarction LV remodeling. In addition, we found improvements in calcium homeostasis, as seen by the protein expression levels of both the RyR and SERCA₂ₐ. This is consistent with the prior clinical findings of improved calcium-handling in DCM patients treated with mechanical support and clenbuterol therapy (Terracciano C M, Harding S E, Adamson D, et al. Changes in sarcolemmal Ca entry and sarcoplasmic reticulum Ca content in ventricular myocytes from patients with end-stage heart failure following myocardial recovery after combined pharmacological and ventricular assist device therapy. Eur Heart J. 2003; 24:1329-39).

This example also demonstrates that clenbuterol therapy led to decreased myocardial apoptosis and increased DNA repair, as seen with quantitative immunohistochemistry staining of key markers of apoptosis, DNA damage, and DNA repair. These improvements may explain the reduction in diastolic LV dysfunction seen in the study with clenbuterol, as myocardial apoptosis has been implicated in the LV remodeling process. Inhibition of apoptosis pathways has also been recently shown to attenuate remodeling (Chandrashekhar Y, Sen S, Anway R, Shuros A, Anand I. Long-term caspase inhibition ameliorates apoptosis, reduces myocardial troponin-I cleavage, protects left ventricular function, and attenuates remodeling in rats with myocardial infarction. J Am Coll Cardiol. 2004; 43:295-301).

The combination of a β-2 adrenergic agonist, clenbuterol, and a β-1 antagonist, metoprolol, did not lead to any evidence of synergy in this study. Although there was evidence of additive improvements in the 8-oxoG and OGG1 staining patterns in the Clen+Meto group, indicating that clenbuterol and metoprolol led to independent improvements in DNA damage and repair, respectively, these effects were not synergistic. It is possible that a synergistic effect would be seen in a different type of study or experiment.

The dosages used for clenbuterol and metoprolol were high-dose, raising the possibility that synergy was not seen with combinational therapy because of the counteraction of β-2 adrenergic effects with the use of an imperfectly selective β-1 antagonist. In addition, the mechanisms by which clenbuterol attenuated diastolic dysfunction were not elucidated by this study.

In summary, this example demonstrates that clenbuterol ameliorates calcium homeostasis, myocardial apoptosis, and EDPVR in a model of ischemic HF. These changes did not have synergy with metoprolol therapy. Trials are underway studying the effects of Clen on cardiac recovery.

Additive effects of β-1 adrenergic antagonism and β-2 stimulation on myocardial apoptotic inhibition and DNA repair in a model of congestive heart failure (Abstract). Circulation 2004 Suppl.

All publications referenced herein are hereby incorporated in their entirety. While the foregoing invention has been described in some detail for purposes of clarity and understanding, it will be appreciated by one skilled in the art, from a reading of the disclosure, that various changes in form and detail can be made without departing from the true scope of the invention in the appended claims.

While the invention has been described in detail with reference to certain embodiments thereof, it will be understood that the invention is not limited to these embodiments. Indeed, modifications and variations are within the spirit and scope of that which is described and claimed.

What is claimed is:

1. A method for treating a patient suffering from congestive heart failure (CHF), not supported by a left ventricle assist device (LVAD), which comprises administering clenbuterol to the patient in an effective amount.

2. The method of claim 1, wherein clenbuterol is administered as clenbuterol hydrochloride.

3. The method of claim 2, wherein clenbuterol hydrochloride is administered as an oral dosage form.

4. The method of claim 3, wherein clenbuterol hydrochloride is administered in daily doses from about 40 mcg to about 4 mg.

5. A method for improving skeletal muscle function in patients suffering from congestive heart failure, not supported by an LVAD, which comprises administering clenbuterol to the patient in an effective amount.

6. The method of claim 5, wherein the clenbuterol comprises clenbuterol hydrochloride.

7. The method of claim 6, wherein clenbuterol hydrochloride is administered as an oral dosage form.

8. The method of claim 7, wherein clenbuterol hydrochloride is administered in daily doses from about 40 mcg to about 4 mg.

9. A method of improving cardiac function in patients with congestive heart failure, not supported by an LVAD, which comprises administering clenbuterol.

10. A method of treating congestive heart failure in patients, not supported by an LVAD, which comprises administering clenbuterol in combination with a beta-1 selective blocker.

11. A method of treating congestive heart failure in patients, not supported by an LVAD, which comprises administering clenbuterol with an ICD.

12. A method of treating congestive heart failure in patients not supported by an LVAD, which comprises administering clenbuterol in combination with a beta-1 selective blocker and an ICD.

13. A method of improving muscle strength as measured in terms of maximal strength or static fatigue index, which comprises administering clenbuterol to patients with congestive heart failure, not supported by an LVAD.

14. A method of improving peak oxygen consumption, peak work or exercise duration during cardiopulmonary exercise testing which comprises administering clenbuterol to patients with congestive heart failure, not supported by an LVAD.

15. A method of improving New York Heart Association Functional Class, which comprises administering clenbuterol to patients with congestive heart failure, not supported by an LVAD.

16. A method of improving patient quality of life according to the Minnesota Living with Heart Failure Questionnaire which comprises administering clenbuterol to patients with congestive heart failure, not supported by an LVAD.

17. A method of lessening symptoms of heart failure, including shortness of breath or fatigue which comprises administering clenbuterol to patients with congestive heart failure, not supported by an LVAD.

18. A method of decreasing hospitalizations for exacerbations of CHF which comprises administering clenbuterol to patients with congestive heart failure, not supported by an LVAD.

19. A method of improving survival for patients with CHF which comprises administering clenbuterol to patients with congestive heart failure, not supported by an LVAD

20. A pharmaceutical composition for use in treating or preventing heart failure comprising a therapeutically effective amount of an adrenergic beta-2 agonist.

21. The pharmaceutical composition of claim 20, wherein the adrenergic beta-2 agonist is selected from the group consisting of albuterol, clenbuterol, formoterol, levalbuterol, metaproterenol, pirbuterol, salmeterol and terbutaline.

22. A pharmaceutical composition for use in treating or preventing heart failure comprising a therapeutically effective amount of an adrenergic beta-1 antagonist in combination with a therapeutically effective amount of an adrenergic beta-2 agonist.

23. The pharmaceutical composition of claim 22, wherein the adrenergic beta-1 antagonist is selected from the group consisting of acebutolol, atenolol, betaxolol, bisoprolol, esmolol and metoprolol, and the adrenergic beta-2 agonist is selected from the group consisting of albuterol, clenbuterol, formoterol, levalbuterol, metaproterenol, pirbuterol, salmeterol and terbutaline.

24. A method for treating or preventing heart failure in a subject, comprising administering to the subject a therapeutically effective amount of an adrenergic beta-2 agonist.

25. The method of claim 24, wherein the adrenergic beta-2 agonist treats or prevents heart failure by treating or preventing cardiac arrhythmia.

26. The method of claim 25, wherein the adrenergic beta-2 agonist treats or prevents heart failure by treating or preventing heart tissue degeneration or reversing the effects of heart failure through the normalization of calcium homeostasis.

27. The method of claim 26, wherein the heart tissue degeneration results from ischemic or non-ischemic causes and in an acute or chronic condition.

28. The method of claim 26, wherein the heart tissue degeneration results from myocardial infarction.

29. The method of claim 24, wherein the effective amount of adrenergic beta-2 agonist is from about 0.01 mg/kg/day to about 2.0 mg/kg/day.

30. The method of claim 24, wherein the adrenergic beta-2 agonist is selected from the group consisting of albuterol, clenbuterol, formoterol, levalbuterol, metaproterenol, pirbuterol, salmeterol, and terbutaline.

31. The method of claim 30, wherein the adrenergic beta-2 agonist comprises clenbuterol.

32. The method of claim 31, wherein the effective amount of clenbuterol comprises from about 0.01 mg/kg/day to about 2 mg/kg/day.

33. A method of treating or preventing heart failure in a subject, comprising administering to the subject a therapeutically effective amount of an adrenergic beta-2 agonist, in combination with a therapeutically effective amount of an adrenergic beta-1 antagonist.

34. The method of claim 33, wherein administration is concurrent.

35. The method of claim 33, wherein administration is sequential.

36. The method of claim 33, wherein administration is alternate.

37. The method of claim 33, wherein a synergistic therapeutic effect results.

38. The method of claim 33, wherein the heart failure is associated with cardiac arrhythmia.

39. The method of claim 33, wherein the heart failure is associated with heart tissue degeneration.

40. The method of claim 33, wherein the heart tissue degeneration results from ischemic or non-ischemic causes and in an acute or chronic condition.

41. The method of claim 33, wherein the heart tissue degeneration results from myocardial infarction.

42. The method of claim 33, wherein the heart tissue degeneration results from a myocardial infarction.

43. The method of claim 33, wherein the effective amount of adrenergic beta-2 agonist is from about 0.01 mg/kg/day to about 2.0 mg/kg/day.

44. The method of claim 33, wherein the adrenergic beta-2 agonist is selected from the group consisting of albuterol, clenbuterol, formoterol, levalbuterol, metaproterenol, pirbuterol, salmeterol, and terbutaline, and the adrenergic beta-1 antagonist is selected from the group consisting of acebutolol, atenolol, betaxolol, bisoprolol, esmolol and metoprolol.

45. The method of claim 33, wherein the adrenergic beta-2 agonist comprises clenbuterol.

46. The method of claim 45, wherein the effective amount of clenbuterol is from about 0.01 mg/kg/day to about 2 mg/kg/day.

47. The method of claim 33, wherein the adrenergic beta-1 antagonist comprises metoprolol.

48. The method of claim 33, wherein the effective amount of adrenergic beta-1 blocker is from about 15 mg/kg/day to about 200 mg/kg/day.

49. The method of claim 44, wherein the effective amount of metoprolol is from about 15 mg/kg/day to about 200 mg/kg/day.

50. A method for preventing heart failure a subject with a pre-heart failure condition, comprising administering to the subject a therapeutically effective amount of an adrenergic beta-2 agonist.

51. A method for preventing heart failure in a subject with a pre-heart failure condition, comprising administering to the subject a therapeutically effective amount of a beta-2 antagonist in combination with a therapeutically effective amount of a beta-1 antagonist.

52. A method for treating or preventing heart failure in a subject post myocardial infarction, comprising administering to the subject a therapeutically effective amount of an adrenergic beta-1 agonist.

53. A method for treating or preventing heart failure in a patient status post myocardial infarction, comprising administering to the subject a therapeutically effective amount of an adrenergic beta-2 agonist.

54. A method for treating or preventing heart failure in a patient status post myocardial infarction, comprising administering to the subject a therapeutically effective amount of an adrenergic beta-2 agonist in combination with a therapeutically effective amount of beta-1 antagonist.

55. A method of treating or preventing heart failure in a subject, comprising administering to the subject an amount of clenbuterol effective to treat or prevent the heart failure, in combination with an amount of an adrenergic beta-1 antagonist effective to reduce the toxicity of clenbuterol.

56. A method of preventing heart tissue degeneration by administering to a subject a therapeutically effective amount of an adrenergic beta-1 antagonist in combination with a therapeutically effective amount of an adrenergic beta-2 agonist.

57. A method for reversing damage to the heart resulting from ischemic or non-ischemic causes, using a combination of an adrenergic beta-1 blocker and an adrenergic beta-2 agonist.

58. A method for reversing damage to the heart following myocardial infarction using a combination of an adrenergic beta-1 blocker and an adrenergic beta-2 agonist.

59. A kit for use in treating and preventing heart failure comprising a combination of an adrenergic beta-1 blocker and an adrenergic beta-2.

60. A kit for use in reversing damage to the heart resulting from ischemic or non-ischemic causes, comprising a combination of an adrenergic beta-1 blocker and an adrenergic beta-2.

61. A kit for use in reversing damage to the heart following myocardial infarction, comprising a combination of an adrenergic beta-1 blocker and an adrenergic beta-2.
